(19) United States
(12) Patent Application Publication
(10) Pub. No.: US 2024/0425825 A1
(43) Pub. Date: Dec. 26, 2024

(54) METHODS FOR PURIFYING FILLED ADENO-ASSOCIATED VIRUS CAPSIDS

(71) Applicant: Regeneron Pharmaceuticals, Inc., Tarrytown, NY (US)

(72) Inventors: Garima Thakur, White Plains, NY (US); Sheldon Robert Mink, Hawthorne, NY (US); Andrew David Tustian, Millwood, NY (US)

(21) Appl. No.: 18/748,663

(22) Filed: Jun. 20, 2024

Related U.S. Application Data
(60) Provisional application No. 63/661,425, filed on Jun. 18, 2024.
(60) Provisional application No. 63/657,389, filed on Jun. 7, 2024.
(60) Provisional application No. 63/654,267, filed on May 31, 2024.
(60) Provisional application No. 63/620,672, filed on Jan. 12, 2024.
(60) Provisional application No. 63/537,158, filed on Sep. 7, 2023.
(60) Provisional application No. 63/522,037, filed on Jun. 20, 2023.

(51) Int. Cl. C12N 7/00

(52) U.S. Cl. CPC C12N 7/00; C12N 2750/14151

(57) ABSTRACT

The inventions provide methods of purifying full adeno-associated virus (AAV) capsids, wherein the method comprises the steps of (a) subjecting a sample comprising AAV capsids in buffer to anion exchange chromatography (AEX) to separate full AAV capsids from capsids that are not full (that is, partially-filled AAV capsids, nearly empty AAV capsids and empty AAV capsids) and form a first pool (“first AEX pass”), wherein the first pool is enriched in the ratio of full capsids to capsids that are not full; and (b) subjecting the first pool to anion exchange chromatography to form a second pool (“second AEX pass”), wherein the second pool is further enriched in the ratio of full capsids to capsids that are not full (that is, partially-filled AAV capsids, nearly empty AAV capsids and empty AAV capsids), wherein the enrichment in full capsids can be least 3 fold. Third, fourth and more AEX passes also can be undertaken. Depth filtration, single-pass tangential flow filtration and affinity exchange also can be included in the inventive methods, preferably undertaken prior to AEX. Enriched AAV preparations and drug products also are provided.

This application claims priority to U.S. Application Ser. No. 63/661,425, filed Jun. 18, 2024; U.S. Application Ser. No. 63/657,389, filed Jun. 7, 2024; U.S. Application Ser. No. 63/654,267, filed May 31, 2024; U.S. Application Ser. No. 63/620,672, filed Jan. 12, 2024; U.S. Application Ser. No. 63/537,158, filed Sep. 7, 2023; and U.S. Application Ser. No. 63/522,037, filed Jun. 20, 2023. These applications are incorporated by reference in their entirety.

### FIELD OF THE INVENTIONS

The present inventions provide methods for purifying filled adeno-associated virus (AAV) capsids. Recombinant AAV of any serotype (rAAV) containing gene(s) of interest (GOI) are increasingly being used in preventative and therapeutic capacities, such as in vaccines and in gene therapy. The increasing use of recombinant AAVs will benefit from improved purification methods.

### BACKGROUND OF THE INVENTIONS

Adeno-associated virus (AAV) is a non-enveloped, single-stranded DNA virus and is used as a gene delivery vector for both research and therapeutics. Weitzman and Linden, Adeno-Associated Virus Biology (chapter 1), Meth. Molec. Biol. 807:1-23 (2011). There are numerous AAV serotypes and variants thereof. AAV serotypes include, for example, AAV1, 2, 3, 4, 5, 6, 7, 8, 9, 10, 11, 12, 13, rh74 as well as variants thereof. See, for example, Issa et al, Cells 12:285 (2023); Goedeker et al., Ther. Adv. Neurol. Disord. 16:1-7 (2023).

Gene transfer vectors based on AAV have demonstrated promise for human gene therapy based on their safety profile and potential to achieve long-term efficacy in animal models. Wang et al., Nature, 18:358-78 (2019). A major challenge for advancing AAV-based therapies into clinical development is the difficulty and cost of producing sufficient quantities of AAV through transient methodologies. Additionally, recombinant AAVs containing genes of interest (GOIs), but lacking surface modifications, are currently being used in preventative and therapeutic capacities, such as in vaccines and in gene therapy. However, such AAVs have limited tissue specificity and a propensity to accumulate in the liver.

Recombinant AAVs (rAAVs) are produced in engineered host cells that contain the requisite genes to allow for the production of the recombinant virus. Recombinant AAV has been produced in HEK 293, BHK, human amniotic (for example, epithelial cells such as HAEpiC), CHO, HeLa and Sf9 lines, for example.

The wild type AAV genome includes a capsid gene referred to as “Cap” or “cap”. Cap in nature is translated to produce, via alternative start codons and transcript splicing, three size-variant structural proteins referred to as VP1 (about 90 kDa), VP2 (about 72 kDa) and VP3 (about 60 kDa). An AAV capsid contains 60 subunits total of the VP proteins. A ratio of 1:1:10 is considered the most typical ratio for VP1: VP2: VP3, with a stoichiometry of 5 VP1 subunits: 5 VP2 subunits: 50 VP3 subunits. However, there can be variation. Wörner et al., Nature Communications 12:1642 (2021). Additionally, production of recombinant AAV results in capsids that are empty, nearly empty or only partially filled in terms of a complete gene of interest and flanking ITRs, which are considered impurities and often are collectively referred to in the field as empty capsids.

A variety of purification approaches need to be employed for rAAV. Depth filtration is used for the purification of AAVs. In current manufacturing processes for rAAV, expensive endonuclease treatment is used prior to depth filtration to break down large amounts of chromatin post-cellular lysis. Without this treatment, traditional depth filters tend to foul rapidly, making clarification impractical without the use of excessive filter area. The present inventions provide for improvements in depth filtration to enhance the efficiency and lower costs of rAAV purification.

Purification of recombinant AAVs, including surface-modified AAVs, also requires effective capture methods. In the context of surface-modified AAVs, challenges in affinity capture include difficulty in binding to the AAV surface epitopes in case of heavy conjugation, and/or difficulty in eluting the surface-modified AAV without destabilizing the molecule leading to aggregation, precipitation, and/or fragmentation. These difficulties often lead to an imbalance in the level of conjugation in the affinity capture load material relative to the eluate stream; that is, a loss of the some or all of the conjugated species during affinity capture due to preferential purification of the less conjugated/unconjugated AAVs. The goal of high-yield affinity capture of rAAVs, including surface-modified (conjugated) AAV species, was not met until the present inventions.

Additionally, separation of full capsids from empty capsids, nearly empty capsids and partially-filled capsids to lessen the presence of these unfilled capsids is an important step in manufacturing of recombinant AAV. Current cell culture technology does not produce only full capsids, that is capsids that contain the entire gene of interest (GOI) and functional flanking AAV inverted terminal repeats (ITRs) required to produce the desired therapeutic effect in vivo. Only a percentage of the rAAV capsids produced in cell culture are full, while the others are partially-filled, nearly empty or empty. It often can be difficult to distinguish between full and partially-filled capsids if the partially-filled capsids are close to full. Likewise, it can be difficult to distinguish between nearly empty and empty capsids if the nearly empty capsids are close to empty. Thus, there are degrees of capsid contents that are less than a complete GOI and flanking ITRs, but nevertheless still are not full. The goal is to enrich for full capsids, and thereby functional capsids, and minimize capsids that are less than full and therefore will not be functional, which is achieved by the present inventions.

Having a high percentage of empty capsids, nearly empty capsids and partially filled capsids in the drug product is undesirable as they contribute to the immune response produced upon drug administration without adding any therapeutic benefit as such capsids do not contain a functional gene of interest and or functional flanking ITRs. Thus, it is critical to enrich for full capsids in the downstream purification process to deliver safer and more efficacious drug products to patients.

There is wide variability in the percentage of full capsids (percent full) produced in the upstream cell culture bioreactor, ranging from 2% to more than 60% full. The percent full (% full) is dependent on a range of factors, including cell line, rAAV serotype, media, feeding strategy, plasmid quality, transfection reagent, transfection kinetics, plasmid design, bioreactor conditions, and process scale. Any changes in the upstream production parameters can therefore impact percent full in the bioreactor. Furthermore, there is often a tradeoff between total viral genome titer (vg/mL) and percent full. For example, novel transfection reagents such as FectoVir® can provide several-fold higher titers than conventional reagents such as polyethyleneimine (such as increasing titers from about 1×10¹⁰ vg/mL to about 1×10¹¹ vg/mL of bioreactor volume), at the cost of several-fold lower % full capsids (such as about 50% full declining to about 5% full). Thus, there is a strong need to create downstream purification processes that can consistently achieve high targets of greater than 70% full capsids despite upstream cell culture process variability.

Currently, there are two primary methods for separation of full and capsids that are not full: density gradient ultracentrifugation based on weight differences, and preparative chromatography based on ion-exchange, metal-ion affinity, or multimodal separations. Density gradient ultracentrifugation can achieve maximum % full enrichment as the weight-based separation is easily able to separate “heavy” DNA-containing full capsids from “light” empty ones. However, the technique is difficult to scale up to a commercial scale as only a few milliliters of material can be processed at one time per centrifuge, with each cycle taking 6 to 12 hours. Thus, preparative ion-exchange chromatography (IEX) is currently the preferred modality for capsid separations in the industry.

However, as IEX relies on differences in surface charge rather than weight, the separation is more challenging as the encapsidated DNA causes only slight differences in the charge, changing the isoelectric point (pl) from about 6.3 for not full capsids to about 5.9 for full capsids. Furthermore, as chromatography separations are achieved by using salt or pH gradients to preferentially displace species, there is a limitation on the enrichment factor that can be achieved, typically ranging from 1.5-to 5-fold. Achieving higher enrichment factors is challenging due to diffusion between the full and not full population peaks during elution. Thus, even a very well-designed IEX method with 3- to 5-fold enrichment is unlikely to be able to achieve greater than, for example, 75% full capsids in case the starting material has a very low percentage of full in the range of 1-30%, and typically 5 to 15%. Furthermore, heterogeneity in not full capsid populations can sometimes shift pl, thereby causing not full capsids to elute on either side of the full peak and necessitating more complex fractionation criteria in the IEX method.

### SUMMARY OF THE INVENTIONS

The inventions provide methods of purifying full adeno-associated virus (AAV) capsids, wherein the method comprises the steps of (a) subjecting a sample comprising AAV capsids to anion exchange chromatography in a buffer using microsteps or linear gradients to separate full AAV capsids from unfilled capsids (that is, partially-filled AAV capsids, nearly empty AAV capsids and empty AAV capsids) and form a first pool (“first pass”), wherein the first pool is enriched in the ratio of full capsids to capsids that are not full; and (b) subjecting the first pool to anion exchange chromatography to form a second pool (“second pass”), wherein the second pool is further enriched in the ratio of full capsids to not full capsids (empty, nearly-empty and partially-filled capsids), wherein the enrichment in full capsids is at least three to five fold. Optionally, a third, fourth and still more passes can be utilized.

The inventions also provide methods of purifying full adeno-associated virus (AAV) capsids, wherein the method comprises the steps of (a) subjecting a sample comprising AAV capsids to depth filtration to form a first pool; (b) subjecting a first pool from step (a) to a purification procedure and then anion exchange chromatography in a buffer using a microstep or a linear gradient to form a second pool, wherein the second pool is enriched in the ratio of full capsids to not full capsids; and (c) subjecting the second pool from step (b) to anion exchange chromatography in a buffer using a microstep or a linear gradient to form a third pool, wherein the third pool is further enriched in the ratio of full capsids to not full capsids. Optionally, the depth filtration can be undertaken without an endonuclease or a reduced amount of an endonuclease.

The inventions further provide methods purifying full adeno-associated virus (AAV) capsids, wherein the method comprises the steps of (a) subjecting a sample comprising AAV capsids to depth filtration to form a first pool; (b) subjecting the first pool from step (a) to affinity chromatography to form a second pool; (c) subjecting the second pool from step (b) to anion exchange chromatography in a buffer using a microstep or a linear gradient to form a third pool, wherein the third pool is enriched in the ratio of full capsids to not full capsids; and (d) subjecting the third pool from step (c) to anion exchange chromatography in a buffer using a microstep or a linear gradient to form a fourth pool, wherein the fourth pool is further enriched in the ratio of full capsids to not full capsids. The term “pool” also encompasses eluents. Optionally, the depth filtration can be undertaken without an endonuclease or a reduced amount of an endonuclease. During depth filtration, the salt content can be less than wherein the salt concentration during depth filtration is less than 100 mM, 75 mM, 50 mM or 25 mM. The salt can be an organic or inorganic salt. Inorganic salts include sodium chloride, potassium chloride, magnesium chloride, calcium chloride, sodium bicarbonate, calcium carbonate, sodium sulfate, calcium phosphate, ammonium chloride, and ammonium sulfate.

The enrichment of full capsids can be 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11 fold or more. The starting samples to be enriched (used in step (a)) typically comprise less than 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, or 30% full AAV capsids.

According to the inventions, the first pool of the method can have 1.5 to 7-fold enrichment in full capsids, for example 30% full to 50% full for a starting load material that is 10% full, for example. The second pool comprises at least 15%, 20%, 25%, 30%, 35%, 40%, 45%, 50%, 60%, 65%, 70%, 75%, 80%, 85%, 90%, 95% or 99% full capsids. The same anion exchange column can be used for the first pass (step (a)) and the second pass (step (b)), and where applicable step (c) also can be on the same anion exchange column. In the alternative, the one or steps can be on different anion exchange columns.

The buffer can comprise at least one buffering agent, for example Bis-Tris-Propane, Bis-Tris, Tris, Glycine, Bicine, Tricine, Acetate, Borate, Citrate, Carbonate, Phosphate, Formate, Sulfate, Succinic acid, Sulfonic acid and variants thereof (for example MES, PIPES, HEPES, CHES, CAPS, MMS, PBMS), Diethanolamine, or Imidazole. The buffer may also comprise one or more organic or inorganic salts, such a sodium chloride, potassium chloride, magnesium chloride, calcium chloride, sodium bicarbonate, calcium carbonate, sodium sulfate, calcium phosphate, ammonium chloride, ammonium sulfate, and tetramethylammonium chloride (TMAC). The buffer may also comprise additives such as antibiotics and bacteriostatics (for example sodium azide or AEBSF), protease inhibitors (for example E64), detergents and chaotropes (for example poloxamer, CHAPS, SDS, Triton, Tween, Urea), saturating agents (for example bovine serum albumin), organic solvents (for example ethanol, isopropanol, acetonitrile), and other additives or excipients including chelatants, stabilizers, reducers (for example DTT, TCEP, EDTA, Glycerol, Sucrose), and amino acids (for example Histidine) The inventions are amenable to the use of anion exchange (AEX) monoliths (for example CIM QA, CIM DEAE, and PRIMA T), AEX resins (for example Capto Q, CAPTO DEAE, POROS HQ, POROS XQ, POROS PI, Fractogel EMD TMAE, Fractogel EMD DEAE, Nuvia Q), and AEX membranes (for example Sartobind STIC PA, Sartobind Q).

According to the inventions, the “first AEX pass” (step (a)) is the first time the AAV sample is loaded and eluted from the AEX chromatographic media, and the “second AEX pass” (step (b)) is the second time the AAV sample is loaded and eluted from the AEX chromatographic media. An optional “third AEX pass” (step (c)) or further passes can be carried out. Elution is based upon a change in ionic strength achieved by mixing two elution buffers (Buffer A and Buffer B) with low and high ionic strength, respectively. The first pass can utilize a series of step changes (“microsteps”) in ionic strength for elution, or gradient elution in which ionic strength is linearly increased from a starting to an ending value (microsteps are preferred). The second and subsequent passes can likewise utilize microsteps or gradient elution. The size of the change in ionic strength in each microstep can be determined from the range of ionic strength in which the full capsids elute in a linear gradient, by dividing the range into parts, such as 2, 3, 4, 5, 6, 7, 8, 9, or 10 parts (2 to 4 parts is preferred). For example, if the full capsids elute in an ionic strength range of 9 mS/cm to 15 mS/cm, corresponding to a difference of 6 mS/cm between the start and end of the range, the size of the microsteps can be 0.6 mS/cm to 3 mS/cm by dividing the range into 2 to 10 parts. The change in ionic strength can be referred to as a percentage change in the change in the percentage of Buffer B mixed with Buffer A. For example, if Buffer A has ionic strength of 1 mS/cm and Buffer B has ionic strength of 20 mS/cm, a microstep of 0.6 mS/cm will correspond to a 3.1% change in Buffer B, while a microstep of 3 mS/cm will correspond to a 15.8% change in Buffer B.

According to the inventions, elution peaks (in the case of microstep elution) or fractions (in the case of gradient elution) where UV260 absorbance is greater than UV280 absorbance at maximum are collected in the first pass. These peak or fraction “elution pools” are loaded again onto the second pass, and so on for subsequent passes The peak or fractions chosen for collection can have UV260/UV280 ratio above a predetermined value, such as 1, 1.05, 1.1, 1.15, 1.2, 1.25, 1.3, 1.35, and 1.4. Typically, UV260/UV280 ratio below 1.1 can contain mostly capsids that are not full and a UV260/UV280 ratio above 1.4 can mostly lack intact AAV capsids.

According to the inventions, elution pools collected in earlier passes can require adjustment in ionic strength in order to be loaded onto subsequent passes, typically to <4 mS/cm and to <2 mS/cm for some AAV types. Typically, ionic strength adjustment is carried out by mixing the elution pools with dilution buffer of low ionic strength, typically of the same composition as elution Buffer A. This can be achieved by pre-filling a bag or collection vessel with dilution buffer and collecting elution pools into the same bag. This can also be achieved via in-line dilution. In another aspect, the requirement of ionic strength adjustment can be eliminated by using a “salt-tolerant” chromatographic medium (monolith, column or membrane) for the second or subsequent passes, where “salt-tolerant” means that the chromatographic medium does not require low ionic strength (typically <4 mS/cm) in the load material to bind loaded material, and can function with AAV load material of ionic strength >10 mS/cm.

According to the inventions, the same chromatographic unit and medium (monolith, column, or membrane) can be used for the first, second, and subsequent passes. Alternatively, a different chromatographic unit or medium can be used for the first, second and subsequent passes. Typically, using the same unit for the first, second and subsequent passes results in highest enrichment, yield and operational simplicity. Two or more units can be used to achieve continuous operation or eliminate the need for in-line dilution by using salt-tolerant chromatographic mediums for the second pass.

According to the inventions, the AAV virus is a recombinant AAV virus comprising a gene of interest flanked by AAV inverted terminal repeats. The gene of interest can encode a protein of interest selected from the group consisting of viral proteins, bacterial proteins, fungal proteins, plant proteins and animal proteins. The gene of interest can encode a human protein. The gene of interest can encode a protein of interest selected from the group consisting of antibodies, receptors, Fc-containing proteins, trap proteins, mini-trap proteins, fusion proteins, antagonists, inhibitors, enzymes, factors, repressors, activators, ligands, reporter proteins, selection proteins, protein hormones, protein toxins, structural proteins, storage proteins, transport proteins, neurotransmitters and contractile proteins. The gene of interest can encode an enzyme that can provide enzyme replacement therapy. The recombinant AAV virus can be used for gene therapy.

According to the inventions, the AAV virus can be a covalently surface modified AAV. The AAV virus capsid can comprise a first member and a second cognate member of a specific binding pair covalently bound together. The second cognate member can be fused to a targeting ligand. The first member and second cognate member can be a system selected from the group consisting of Spy Tag: Spy Catcher, Spy Tag002: SpyCatcher002, SpyTag003: SpyCatcher003, SnoopTag: SnoopCatcher, Isopeptag: Pilin-C, Isopeptag: Pilin-N, SnoopTagJr: SnoopCatcher, DogTag: DogCatcher, SdyTg: SdyCatcher, Jo: In, 3kptTag: 3kptCatcher, 40q1Taq/4oq1 Catcher, NGTag/NGCatcher, Rumtrunk/Mooncake, Snoop ligase, GalacTag, Cpe, Ececo, and Corio. Spy Tag002: SpyCatcher002 and SpyTag003: SpyCatcher003 are different iterations of Spy Tag: Spy Catcher.

The inventions also provide an enriched AAV preparation produced by any of the above the methods, and drug products made from the enriched AAV preparations.

Advantages provided by the inventions and optional for the skilled person, include but are not limited to, (1) microsteps and linear gradients to facilitate collection of full capsids in the first pass with UV based collection criteria, (2) methodologies to determine the size of the microsteps that works across AAV serotypes, variants, chromatographic units and buffer systems by converting a linear gradient into a series of microsteps; (3) achievement of high enrichment factors for full AAV capsids even with very low starting % full capsids that are not achievable in single pass; (4) Provision of both microstep to step and microstep step to gradient options; (5) automation and scale-up of the method on unitary systems with a single chromatography unit with in-line dilution; and (6) integration with salt-tolerant chromatography units for the second pass and conversion to continuous processing by using two or more chromatography units in series.

The inventions also can advantageously single-pass tangential flow filtration, preferably after depth filtration and before affinity capture and/or anion exchange. For example, the inventions provide methods of purifying full adeno-associated virus (AAV) capsids, wherein the method comprises the steps of (a) subjecting a sample comprising AAV capsids to depth filtration to form a first pool; (b) subjecting a first pool from step (a) to a single-pass tangential flow filtration to form a retentate comprising AAV capsids; (c) subjecting the retentate to affinity capture to form a second pool comprising AAV capsids; (d) subjecting the second pool to anion exchange chromatography in a buffer using a microstep or a linear gradient to form a third pool, wherein the third pool is enriched in the ratio of full capsids to capsids that are not full; and (e) subjecting the third pool from step (d) to anion exchange chromatography in a buffer using a microstep or a linear gradient to form a fourth pool, wherein the fourth pool is further enriched in the ratio of full capsids to capsids that are not full. The depth filtration optionally does not require an endonuclease. The single-pass tangential flow filtration can use a permeate pump. The affinity capture can further comprises non-AAV viral inactivation.

Tangential flow filtration (TFF) is a generic term and includes, but is not limited to, ultrafiltration/diafiltration (UF/DF) and newer approaches such as single-pass tangential flow filtration (SPTFF).

The inventions are amenable to all AAV serotypes including, for example, AAV1, AAV2, AAV2quad (Y-F), AAV3, AAV4, AAV5, AAV6, AAV7, AAV8, AAV9, AAV10, AAV11, AAV12, AAV13, rh10, rh39, rh43, rh74, Avian AAV, Sea Lion AAV, Bearded Dragon AAV, as well as variants thereof.

### BRIEF DESCRIPTION OF THE FIGURES

FIGS. 1A and 1B provide data on filtrate quality analytics for Harvest RC (HRC) and COSP filtration of rAAV8 (3×10⁶ cells/ml) and rAAV9 (1.5×10⁶ cells/ml). FIG. 1A is a bar graph for AAV8 depicting in order bars for bioreactor lysate (white bars), filtrate pool where transmembrane pressure (TMP) is 5 psi (light gray bars), and filtrate pool where TMP is 15 psi (black bars). HRC (solid bars) and C0SP (a standard filter used as a control) (hatched and labeled bars) are filtration trains with different endonuclease and salt conditions in the load. FIG. 1B is a bar graph for AAV9 depicting in order bars for bioreactor lysate (white bars), filtrate pool where TMP is 5 psi (light gray bars), and filtrate pool where TMP is 15 psi (black bars). HRC (solid bars) are filtration trains with different endonuclease and salt conditions in the load. In FIG. 1A, significant breakthrough of HCDNA into the filtrate was observed for COSP at the “COSP Negative Control”, and for HRC at 250 mM NaCl and 0 U/ml endonuclease (dark gray bars). In FIG. 1B, significant breakthrough of HCDNA into the filtrate was observed for HRC at 250 mM NaCl and 0 U/ml endonuclease (dark gray bars) and 250 mM NaCl and 10 U/ml endonuclease (dark gray bars).

FIGS. 2A-2F provide data for depth filtration treatment at various salt and endonuclease conditions. FIGS. 2A-2C provide data from testing of AAV8 and FIGS. 2D-2F provide data from testing of AAV9.

FIG. 3 depicts data set forth in FIGS. 2A-2F. Throughputs for endonuclease at the 10 U/ml condition were midway between the 0 U/ml and 100 U/ml endonuclease conditions.

FIG. 4 depicts data set forth in FIGS. 2A-2F regarding the effects of salt.

FIG. 5 depicts data showing that salt addition reduces load turbidity and increases throughput at higher pressures.

FIG. 6 depicts data set forth in FIGS. 2A-2F showing that a combined cake-fiber fouling model is a good representation for the mechanism of Harvest RC filtration (depth filtration).

FIGS. 7A-7E concern the effects of endonucleases. FIGS. 7A-7C depict data set forth in FIGS. 2A-2F. Cake formation parameters (K_{c}) vs NaCl (mM) added with 0 U/ml endonuclease (FIG. 7A), 10 U/ml endonuclease (FIG. 7B), and 100 U/ml endonuclease (FIG. 7C). FIG. 7D depicts filtration performance at 50 L scale that was evaluated with rAAV9 and rAAV8. FIG. 7E is a chart comparing the performance of Harvest RC and COSP in the bioreactors and the clarified pool at endonuclease concentrations of 100 U/ml or 0 U/ml.

FIG. 8 depicts data using a POROS CaptureSelect AAV9 resin to capture AAV9-SpyT-SpyC-mAb. Monoclonal antibodies against CACNG1, ASGR1 and Feld1 were used as retargeting molecules. Less conjugated AAV9 (1/20 and 1/30) achieved higher yields.

FIGS. 9A-9B concern columns and buffers. FIG. 9A compared POROS CaptureSelect AAV9 and POROS CaptureSelect AAV X using glycine or citrate elution buffers. FIG. 9B compared various buffers used for affinity capture of an AAV-SpyT-SpyC-Tfr Fab. A Poros CaptureSelect AAV9 column loaded with 10¹³ to 10¹⁵ capsids per milliliter was employed. The yield of the affinity capture step was heavily dependent on the choice of elution buffer. Low yields were achieved in many elution buffers along with loss of the heaviest conjugated species, whereas certain buffers were able to achieve yield >90% along with successful capture of the heavily-conjugated AAV species.

FIG. 10 illustrates a two-step affinity capture protocol utilizing (i) affinity to AAV surface epitopes and (ii) affinity to the antibody surface epitopes. The two affinity capture steps can be employed in any order. Resins used for affinity capture via AAV surface epitopes can comprise POROS CaptureSelect AAVX, POROS CaptureSelect AAV8, POROS CaptureSelect AAV9, Capto AVB, AVB Sepharose, Avipure AAV2, Avipure AAV8, Avipure AAV9, among others. Resins used for affinity capture via antibody surface epitopes can comprise mAbSelectSuRe, mAbSelect PrimaA, Capto L, mAbSelect VL, KappaSelect, among others. The two-step capture protocol allows effective removal of unconjugated antibody as well as unconjugated AAV, resulting in an eluate pool with only conjugated AAV.

FIG. 11 is a bar graph depicting data relating to the selection of SpyTag fraction and SpyCatcher mAb DNA concentration and a description on the right. Spy Tag fractions (pRC-SpyT to pRC) ranged from 1/5 to 1/30. The percent change in mAb DNA concentration ranged 0% to 200% Lane 5, which had a 1/10 SpyTag fraction and a 100% increase in mAb DNA showed the highest transduction efficiency. The signal was generated using a green fluorescent reporter gene.

FIGS. 12A-12H are as follows: FIGS. 12A-12C describe different setups that can be used to carry out two-pass anion exchange purification described of these inventions. FIG. 12A describes the setup for two or more passes on the same chromatography unit. FIG. 12B describes the setup for two passes on different chromatography units which can be used for continuous operation. FIG. 12C describes the setup for two passes with the second pass on a salt-tolerant chromatographic unit eliminating the need for in-line dilution. There will exist empty and partially-filled capsid populations, some of which can have isolectric points (pl) that are above the pl of the full capsids, and others that can have isolectric points that are below the pl of the full capsids. FIG. 12D is a graph depicting the elution of empty capsids and full capsids using an increasing salt gradient for AAV2 and AAV8, where empty capsids elute before the full capsids. FIG. 12E depicts exemplary elution profiles for AAV9 and AAV9-SpyT for empty, partially filled and full capsids, where a small population of empty capsids elutes before the full capsids, and a larger population of empty capsids elutes after the full capsids. These two profiles in FIGS. 12D and 12E serve as a model for other AAVs. FIG. 12F is a graph depicting separation empty, partially filled and full AAV9-SpyT capsids using a hybrid gradient combining linear and step gradients. The column was a CIM QA monolith loaded with 5×10¹³ to 5×10¹⁴ capsids (cp/ml). The buffer was 20 mM Bis-Tris-Propane, 0.001% P188, pH 9.6 and 1M NaCl and the process resulted in enrichment of full capsids from 14% to 55%. This hybrid gradient enabled improved resolution of the full capsids (central peak) from the empty capsid populations on either side. However, the hybrid gradient is operationally challenging at scale and also does not result in full capsids above the preferred threshold of 70%. FIGS. 12G and 12H depict fluorescence emission intensity profiles (lower graphs) along with UV280 and UV260 profiles (upper graphs) for AAV2 (FIG. 12G) and AAV9 (FIG. 12H). The fluorescence intensity is a measure of true capsid concentration as it is unaffected by the presence or absence of DNA inside the capsids. The profiles show that the empty capsid peaks appear smaller than the full capsid peaks when measured by UV, but their true size is evident when using fluorescence, showing that empty capsids are more abundant than full capsids. The “overlap” region between the empty and full capsid peaks indicates suboptimal separation using linear gradients alone.

FIGS. 13A-13D provide a comparison of percent full achieved by one AEX pass and two AEX pass methods using rAAV9. FIG. 13A shows results from a one AEX pass linear grade elution. FIG. 13B shows results from a one AEX pass elution using an optimized linear step gradient. FIG. 13C shows results from the first AEX pass of a two AEX pass method. FIG. 13D shows the results of the second AEX pass of a two AEX pass method. FIG. 13A used a 1 ml CIM QA monolith column. FIGS. 13B-13D used 400 ml CIM QA monolith column (from 2×50 L productions). 5×10¹³ capsids/ml were use on all runs. “Full” capsids are considered functional in terms of a complete GOI and flanking ITRs, whereas “partial” or “partially-filled” capsids would not.

FIGS. 14A-14E provide a comparison of percent full achieved by one AEX pass and two AEX pass methods using rAAV2. FIG. 14A is a graph depicting elution on a CIM QA Linear Gradient. FIG. 14B is a graph depicting a CIM QA First AEX Pass. FIG. 14C is a CIM QA Second AEX Pass using the pool from FIG. 14B. FIG. 14D is a CIM QA Third AEX Pass using the pool from FIG. 14C. FIG. 14E is a Second AEX Linear Gradient Pass using the pool of FIG. 14B.

FIGS. 15A-15E provide a comparison of percent full achieved by one AEX pass and two AEX pass methods using rAAV9-Spy Tag. FIG. 15A is a graph depicting elution on a CIM QA Linear Gradient. FIG. 15B is a graph depicting a CIM QA First AEX Pass. FIG. 15C is a CIM QA Second AEX Pass using the pool from FIG. 15B. FIG. 15D is a CIM QA Third AEX Pass using the pool from FIG. 15C. FIG. 15E is a Second AEX Linear Gradient Pass using the pool of FIG. 15B.

FIGS. 16A-16D provide a comparison of percent full achieved by one AEX pass and two AEX pass methods using rAAV9-Spy Tag-Spy Catcher-FELD1 Ab. FIG. 16A is a graph depicting elution on a Prima T Linear Gradient. FIG. 16B is a graph depicting a Prima T Second AEX Pass. FIG. 16C is a Prima T Third AEX Pass using the pool from FIG. 16B. FIG. 16D is a Prima T Second Linear Gradient AEX Pass using the pool from FIG. 16A.

FIGS. 17A-17D provide another comparison of percent full achieved by one AEX pass and two AEX pass methods using rAAV2. FIG. 17A is a graph depicting elution on a CIM QA Linear Gradient. FIG. 17B is a graph depicting a CIM QA First AEX Pass. FIG. 17C is a CIM QA Second AEX Pass using microsteps using the pool from FIG. 17B. FIG. 17D is a CIM QA Second AEX Pass using linear gradient the pool from FIG. 17B. Mass photometry histograms depicting populations of full and empty capsids are displayed for the load, first pass pool, and second pass pools. “EC” stands for an “Empty Capsid,” also referred to as “Empty.” “VC” stands for “Full Viral Capsid,” also referred to as “Full.”

FIG. 18A depicts data from a two-pass anion exchange chromatogram for a 500 L purification of AAV9-SpyTag using first pass and second pass microsteps. This process achieved a recovery of 96% full capsids. Partially-filled capsids were only 1% of total and empty capsids were only 3% of total. FIG. 18B depicts initial separation of bulk empty capsids in a first AEX pass followed by further removal of empty capsids in an AEX second pass. FIG. 18C depicts mass photometry data showing an enrichment from 36% full capsids to 81% full capsids after a first AEX pass to 98% full capsids after a second AEX pass. FIG. 18D depicts small scale purification of rAAV1 (1 ml scale) with histograms on top and mass photometry chromatograms below, where the first pass of AEX resulted in an enriched pool of full rAAV1 capsids and the second pass of AEX resulted in a further enriched pool of full rAAV1 capsids. FIG. 18E depicts a pilot scale chromatogram of rAAV1 in a 50 liter bioreactor using an automated two-pass Akta AEX Pilot 600 system using 400 ml of a CIM QA HR monolith.

FIG. 19 depicts a comparison of percent full achieved by one AEX pass and two AEX pass methods using AAV2, AAV8 and AAV9-SpyT on CIM QA monolith, POROS 50 HQ resin, and Sartobind Q membrane chromatographic modalities. The target of greater than 70% full capsids is achieved for all three serotypes using CIM QA monolith, and also on Poros 50 HQ resin for AAV9-SpyT, but not using Sartobind Q membranes for AAV9-SpyT.

FIGS. 20A-E are as follows: FIG. 20A depicts an automated approach for a two AEX pass method using Atka Avant; Akta Pilot and Akta Ready Extended systems, using microsteps for the first pass and a gradient elution for the second pass. The left side of the graph depicts the first load and first pass elution (collectively “the first AEX pass”) and the right side of the graph shows second pass load and the second pass elution (collectively “the second AEX pass”), while the table illustrates the pump inlet and system outlet connections required to achieve the automated two-pass method. FIG. 20B illustrates a method design for an automated two-pass AEX method using microsteps for both the first and second passes on an Atka Pilot 600 system. FIG. 20C depicts a two-pass AEX chromatogram for AAV2 carried out at 50 L bioreactor scale on a 400 mL CIM QA monolith, resulting in a final pool with greater than (>) 70% full capsids. FIG. 20D depicts a two-pass AEX chromatogram for AAV8 carried out at 50 L bioreactor scale on a 80 mL CIM QA monolith, resulting in a final pool with >70% full capsids. FIG. 20E depicts a two-pass AEX chromatogram for AAV9-SpyT at 500 L bioreactor scale on a 400 mL CIM QA monolith, resulting in a final pool with 96% full and less than 1% partial capsids as measured by mass photometry.

FIG. 21 depicts production purification trains. The top train uses a batch tangential flow filtration unit where repeated passes are required to exchange buffer and concentrate the retentate. The bottom section replaces the batch tangential flow filtration unit with a single pass tangential flow filtration unit. Ionic exchange chromatography of different modalities can be used following TFF, and anion exchange is depicted as an exemplar.

FIG. 22A schematically shows a batch tangential flow filtration (Batch TFF) (top), where the retentate is repeatedly cycled through a feed tank and pump to repeatedly passed through a membrane, with the concentrated permeate being removed after repeated cycles. A single pass tangential flow unit (Single-Pass TFF or SPTFF) (bottom) removes material from the feed tank through a pump to a multi-stage membrane module that separate the retentate from the permeate, while concentrating the permeate. FIG. 22B is a graph comparing Batch TFF and Single-Pass TFF. Single-Pass TFF achieves higher concentration and is faster as compared to Batch TFF. Single-Pass TFF continuously delivers biological material (such as AAV) to the next operation in the purification train, whereas Batch TFF does not deliver biological material (such as AAV) until the end of the batch cycle.

FIG. 23 schematically compares the batch operation to a continuous operation in terms of Cell lysis, Clarification, TFF (Batch or Single-Pass) and Affinity Capture. The continuous process can be completed in less than a day, whereas the batch process can be multi-day.

FIG. 24 schematically depicts exemplary arrangements for multi-stage membrane module cassettes to be used with Single-Pass TFF. The configurations depict four to seven tiers of membrane module cassettes where the initial tiers (left side) contain more or same number of membrane module cassettes as the succeeding tiers (moving towards the right side). Total area and path length of the membrane module cassettes also are set forth.

FIG. 25 is a graph depicting volumetric concentration factor (VCF) versus transmembrane pressure (TMP) using the 4-in-series, 5-in-series, 6-in-series and 7-in-series exemplary configurations depicted in FIG. 24 with a feed comprising an exemplary AAV, here AAV9 comprising a SpyTag insert.

FIG. 26 depicts data from a 5-in-series configuration according to FIG. 24 at flow rates of 90 ml/minute, 120 ml/minute and 150 ml/minute. The log best-fit equation of VCF=A In (TMP-B) using the values at each flow rate set forth near the plot (and rounded off in the chart) can be used to parameterize the data. At the right side of the figure, there is a graph of parameter value (A, B) and feed flow rate in liters per square meter of membrane per hour (LMH) for 4-in-series and 5-in-series exemplary configurations of FIG. 24 and allows optimized conditions to be selected in silico using an exemplary AAV, here AAV9 comprising a SpyTag insert. This model can be used to predict the VCF for any flow rate and TMP for an in-series configuration of interest.

FIG. 27A is a design space model based on FIGS. 22A, 22B and 23 using the 5-in-series configuration of FIG. 24. Here, the process target was 35 LMH, and the intersecting lines indicate a VCF of 8 and a TMP of 10 psi. An exemplary acceptable zone would be a VCF of 6-10 and a TMP of 7.5 to 12.5 psi. FIG. 27B is an exemplary comparison of process parameters between SPTFF and Batch TFF. With Batch TFF, typically there would be one batch before the next operation. However, depending on the scheduling of upstream production bioreactors and bioreactor titers, there could be pooling of multiple batches before the next operation

FIG. 28 depicts data from a bench-scale trial to determine the number of buffer washes need to attain about a 90% recovery of AAV, here AAV9 with integrated SpyTag, in a low-TMP process. On average, the AAV9 here contained an average of 6 SpyTag peptides per capsid. Capsid titer in retenate (cp/ml) versus SPTFF operating time (minutes) was measured using four buffer flushes. As the right side of the figure shows, it was determined that only two buffer flushes were required to achieve about a 90% recovery with a VCF of 8×.

FIG. 29 is a graph depicting Permeate Flux (LMH), Throughput (L/m²), Feed Flow Rate (L/hr) and TMP (psi) in a pilot-scale trial. The data showed flux decline and TMP build up. To mitigate TMP increase beyond 12.5 psi, feed flow rate was slowed. This resulted in a longer process time of 180 minutes rather than the expected 90 minutes and an overall VCF of 5× was achieved rather than the target of 8×.

FIG. 30 depicts a tween micelle build-up on the TFF membrane, which is believed to be the cause of an unexpected flux decline of about 50%. This figure also set forth the approximate size of AAV, Host Cell protein aggregates (HCP) and Tween-20 micelles. Detergents, such as Tweens, are a common component of cell lysis buffers used in the purification of AAV.

FIG. 31 is a graph depicting fold presence of Tween-20 on the retentate side of membrane and the Permeate side of the membrane for both Batch TFF and SPTFF.

FIG. 32 is a graph depicting the flux decline after two hours with varying percentages of Tween-20 in the lysis buffer. In addition to Tween-20, the buffer contained 20 mM Tris, 2 mM MgCl₂ at a pH of 7.4. The feed flow rate was 35 LMH and the TMP was about 5 to 10 psi.

FIG. 33 compares control with the retentate valve to control with a permeate pump. Option 1 with the retentate valve found that TMP reached 22 psi, and after which the flow had to be reduced from 40 LMH to 30 LMH. VCF dropped from about 10× to about 6×. Option 2 with the permeate pump was superior. TMP was controlled to well under 10 psi and a VCF of 8× was maintained. At the right side of the figure, Option 1 (SPTFF with retentate valve) and Option 2 (SPTFF with permeate pump) were compared to a Batch TFF. Option 1 did not perform as well as Option 2 and Batch TFF. Option 2 was superior to Batch TFF and Option 1 in terms of capsid yield and percent aggregation.

FIG. 34 depicts the overall pilot scale process, and has similarities to parts of the production process of FIG. 21.

FIG. 35 compares VCFs (1-14), SPTFF retentate flow rates and residence time in affinity capture. VCFs of 7 to 13 and SPTFF retentate flow rates of 75-40 provided an exemplary range of residence time suitable for affinity loading.

FIG. 36 depicts how UV280 profile of affinity capture flow can be used for process monitoring of VCF and process stability using SPTFF for continuous processing. Three different runs were performed for comparison purposes. Run 1 was performed without a permeate pump and achieved a VCF of only 5×. Run 2 was performed with a permeate pump with a feed to retentate flush (with recirculation) and achieved a VCF of 8×. Run 3 was performed with a permeate pump with a feed to retentate flush (with recirculation) and a permeate to retentate flush and achieved a VCF of 10×.

### DETAILED DESCRIPTION OF THE INVENTIONS

### Definitions

Unless defined otherwise, all technical and scientific terms used herein have the same meaning as commonly understood by one of ordinary skill in the art to which this invention belongs.

The term “about” in the context of numerical values and ranges refers to values or ranges that approximate or are close to the recited values or ranges such that the invention can perform as intended, such as having a desired rate, amount, density, degree, increase, decrease, percentage, value, purity, pH, concentration, presence of a form or variant, temperature or amount of time, as is apparent from the teachings contained herein. For example, “about” can signify values either above or below the stated value in a range of approx. +/−10% or more or less depending on the ability to perform. Thus, this term encompasses values beyond those simply resulting from systematic error.

A “gene of interest” (GOI) encodes a “protein of interest” or “polypeptide of interest” and optionally can include other associated sequences. The sequences can be natural, semi-synthetic or synthetic. Native sequences, mutant sequences and degenerate sequences can be GOIs. A gene of interest also can be referred to as a “transgene” or a “polynucleotide of interest.”

“Protein of interest” or “polypeptide of interest” (POI) can have any amino acid sequence, and includes any protein, polypeptide, or peptide that is desired to be expressed. Included are, but not limited to, viral proteins, bacterial proteins, fungal proteins, plant proteins and animal (including human) proteins. Protein types can include, but are not limited to, antibodies, receptors, Fc-containing proteins, trap proteins (including mini-trap proteins), fusion proteins, antagonists, inhibitors, enzymes (such as those used in enzyme replacement therapy), factors, repressors, activators, ligands, reporter proteins, selection proteins, protein hormones, protein toxins, structural proteins, storage proteins, transport proteins, neurotransmitters and contractile proteins. Derivatives, components, domains, chains and fragments of the above also are included. The sequences can be natural, semi-synthetic or synthetic.

“Purification” in its various grammatical forms includes, but is not limited to, the use of one or more procedures, such as depth filtration, tangential flow filtration, affinity capture, ionic exchange (such as anionic exchange and cationic exchange) and the like.

The term “recombinant capsid protein” includes a capsid protein that has at least one mutation in comparison to the corresponding capsid protein of the wild-type virus, which wild-type may be a reference and/or control virus for comparative study. A recombinant capsid protein includes a capsid protein that comprises a heterologous amino acid sequence, which may be inserted into and/or displayed by the capsid protein. “Heterologous” in a general context means heterologous as compared to the virus, from which the capsid protein is derived. The inserted amino acids can simply be inserted between two given amino acids of the capsid protein. An insertion of amino acids can also go along with a deletion of given amino acids of the capsid protein at the site of insertion, for example, 1 or more capsid protein amino acids are substituted by 5 or more heterologous amino acids). An example of a heterologous amino acid sequence that can be inserted is a member of a specific binding pair, such SpyTag.

“Retargeting” or “redirecting” may include a scenario in which the wildtype vector targets several cells within a tissue and/or several organs within an organism, which general targeting of the tissue or organs is reduced to abolished by insertion of the heterologous epitope, and which retargeting to more a specific cell in the tissue or a specific organ in the organism is achieved with the targeting ligand that binds a marker expressed by the specific cell. Such retargeting or redirecting may also include a scenario in which the wildtype vector targets a tissue, which targeting of the tissue is reduced to abolished by insertion of the heterologous epitope, and which retargeting to a completely different tissue is achieved with the targeting ligand.

“Detargeting” refers to reducing or abolishing AAV natural preferential transduction by mutating Cap proteins. For example, mutations in the galactose binding domain of VP1 assist in detargeting the liver.

By way of example, different AAV serotypes are known to preferentially transduce the cells of different tissues. Tissue specificity is limited, and AAV is known to preferentially transduce the liver, which can be a safety and efficacy concern in some contexts. The inventions further provide mutations in the VP1 Protein of AAV9 to lower the AAV preferential transduction of the liver. The mutations include N272A and W503A substitutions, where alanine replaces both asparagine at position 272 of VP1 and tryptophan at position 503 of VP1. One or both of the mutations can be undertaken in the VP1 protein. Optionally, other amino acids, such as glutamic acid, serine or others, can be used instead of alanine for substitution. Other detargeting mutation sites include, but are not limited to, N470, D271, and Y446. The inventions provide exemplary mutations for other AAVs are as follows: AAV1-N500E; AAV2-R585A and R588A; AAV5-T571S AAV6-N500E, K531A and K531E. These and others are set forth in the chart below:

| AAV | | |
|---|---|---|
| serotype | Insertion Sites | Exemplary Mutations |
| AAV2 | 453, 587, 1, 34, 138, 139, 161, 261, 266, | R484, R487, R585A, R588A and K532 |
| | 381, 447, 448, 459, 471, 520, 534, 570, | R484A, R487A, R487G, K532A, K532D, |
| | 573, 584, 588, 591, 657, 664, 713, 716 | R585A, R585S, R585Q, R588A, R588T |
| AAV9 | 453, 587, 589 | N272A, W503A |
| AAV1 | 587, 589 | N500E |
| AAV3 | 585 | |
| AAV4 | 584, 585 | |
| AAV5 | 575, 585 | K531A, K531E, T571S |
| AAV6 | | N500E, K531A, K531E |
| Avian AAV | 444, 580 | |
| Sea lion | 429, 430, 431, 432, 433, 434, 436, | |
| AAV | 437, 565 | |
| Bearded | 573, 436 | |
| Dragon AAV | | |

Still other mutations for all AAV serotypes are available to the person skilled in the art.

The term “retargeting molecule” is a molecule useful for targeting an antigen, receptor and/or ligand found on the surface of a cell. The retargeting molecule is bound to a polypeptide that is part of a specific binding pair. For example, a targeting molecule could be bound to SpyCatcher in order to utilize the SpyTag-SpyCatcher system. The retargeting molecule can target the cell that has the antigen, receptor and/or ligand that the retargeting molecule can bind to, and thereby direct a recombinant AAV to that cell. Fc-containing proteins, such as antibodies, monoclonal antibodies (including derivatives, fragments, half antibodies and other heavy chain and/or light chain combinations), multispecific antibodies (for example, bispecifics, IgG-ScFv, IgG-dab, ScFV-Fc-ScFV, trispecifics), Fc-fusion proteins, receptor-Fc fusion proteins, trap proteins and mini-trap proteins, are useful as retargeting molecules.

All antibody classes, namely IgG, IgA, IgM, IgD and IgE, can be used as retargeting molecules. IgG is a preferred class, and includes subclasses IgG1 (including IgG1) and IgG1K), IgG2, IgG3, and IgG4. Further antibody types include a human antibody, a humanized antibody, a chimeric antibody, a monoclonal antibody, a multispecific antibody, a bispecific antibody, a trispecific antibody, an antigen binding antibody fragment, a single chain antibody, a diabody, triabody or tetrabody, a Fab fragment or a F(ab′)λ fragment, an IgD antibody, an IgE antibody, an IgM antibody, an lgG antibody, an IgG1 antibody, an IgG2 antibody, an IgG3 antibody, or an IgG4 antibody.

“Specific binding pair,” “protein: protein binding pair” and the like includes two proteins (that is, a first member, such as a first polypeptide, and a second cognate member, such as a second polypeptide) that interact to form a covalent isopeptide bond under conditions that enable or facilitate isopeptide bond formation, wherein the term “cognate” refers to components that function together by to reacting together to form an isopeptide bond. Thus, two proteins that react together efficiently to form an isopeptide bond under conditions that enable or facilitate isopeptide bond formation can also be referred to as being a “complementary” pair of peptide linkers. Specific binding pairs capable of interacting to form a covalent isopeptide bond are reviewed in Veggiani et al. (2014) Trends Biotechnol. 32:506, and include, for example, peptide: peptide binding pairs such as Spy Tag: Spy Catcher,
SpyTag002: SpyCatcher002, SpyTag: KTag, isopeptag: pilin C,
SnoopTag: SnoopCatcher and others. Spy Tag002: SpyCatcher002 and
SpyTag003: SpyCatcher003 are different iterations of Spy Tag: Spy Catcher.

The term “isopeptide bond” refers to an amide bond between a carboxyl or carboxamide group and an amino group at least one of which is not derived from a protein main chain or alternatively viewed is not part of the protein backbone. An isopeptide bond may form within a single protein or may occur between two peptides or a peptide and a protein. Thus, an isopeptide bond may form intramolecularly within a single protein or intermolecularly, that is between two peptide/protein molecules, such as between two peptide linkers. Typically, an isopeptide bond may occur between a lysine residue and an asparagine, aspartic acid, glutamine, or glutamic acid residue or the terminal carboxyl group of the protein or peptide chain or may occur between the alpha-amino terminus of the protein or peptide chain and an asparagine, aspartic acid, glutamine or glutamic acid. Each residue of the pair involved in the isopeptide bond is referred to herein as a reactive residue. An isopeptide bond may form between a lysine residue and an asparagine residue or between a lysine residue and an aspartic acid residue. Particularly, isopeptide bonds can occur between the side chain amine of lysine and carboxamide group of asparagine or carboxyl group of an aspartate.

The term “peptide tag” includes polypeptides that are (1) heterologous to the protein which is tagged with the peptide tag, (2) a member of a specific protein-protein binding pair capable of forming an isopeptide bond, and (3) no more than 50 amino acids in length.

The term “target cells” includes any cells in which expression of a nucleotide of interest is desired. Preferably, target cells exhibit a target, such as a receptor, ligand and/or antigen on their surface that allows the cell to be targeted. Exemplary targets are calcium voltage-gated channel auxiliary subunit gamma 1 (CACNG1), asialoglycoprotein receptor 1 (ASGR1), Fel d 1, ENTPD3, PTPRA, CD20, CD63 and Her2. Additional targets include GAB A, transferrin. CD3, CD34, integrin, adipophilin, AIM-2, ALDHIAI, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNKIAI, CTAGI, CTAG2, cyclin DI, Cyclin-A1, dek-can fusion protein, DKK1, EFTUD2, Elongation factor 2, ENAH (hMena), Ep-CAM, EpCAM, EphA3, epithelial tumor antigen (“ETA”), ETV6-AML1 fusion protein, EZH2, E6, E7, FGF5, FLT3-ITD, FN1, G250/MN/CAIX, GAGE-1,2,8, GAGE-3,4,5,6,7, GAS7, glypican-3, GnTV, gpIOO/Pmel 17, GPNMB, HAUS3, Hepsin, HER-2/neu, HERV-K-MEL, HLA-A11, HLA-A2, HLA-DOB, hsp70-2, IDOI, IGF2B3, IL13Ralpha2, Intestinal carboxyl esterase, K-ras, Kallikrein 4, KIF20A, KK-LC-1, KKLC1, KM-HN-1, KMHN1 also known as CCDCl10, LAGE-1, LDLR-fucosyltransferase AS fusion protein, Lengsin, M-CSF, MAGE-A1, MAGE-A 10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, ME1, Mel an-A/MART-1, Meloe, Midkine, MMP-2, MMP-7, MUC1, MUC5AC, mucin, MUM-1, MUM-2, MUM-3, Myosin, Myosin class I, N-raw, NA88-A, neo-PAP, NFYC, NY-BR-1, NY-ESO-I/LAGE-2, OA1, OGT, OS-9, P polypeptide, p53, PAP, PAX5, PBF, pml-RARalpha fusion protein, polymorphic epithelial mucin (“PEM”), PPP1R3B, PRAME, PRDX5, PSA, PSMA, PTPRK, RAB 38/N Y-MEL-1, RAGE-1, RBAF600, RGS5, RhoC, R F43, RU2AS, SAGE, secernin 1, SIRT2, SNRPD1, SOX10, Spl7, SPA17, SSX-2, SSX-4, STEAPI, survivin, SYT-SSX1 or -SSX2 fusion protein, TAG-1, TAG-2, Telomerase, TGF-betaRII, TPBG, TRAG-3, Triosephosphate isomerase, TRP-I/gp75, TRP-2, TRP2-INT2, tyrosinase, tyrosinase (“TYR”), VEGF, WT1, XAGE-lb/GAGED2a, Kras, NY-ESOI, MAGE-A3, HPV E2, HPV E6, HPV E7, WT-1 antigen (in lymphoma and other solid tumors), ErbB receptors, Melan A [MARTI], gp 100, tyrosinase, TRP-I/gp 75, and TRP-2 (in melanoma); MAGE-1 and MAGE-3 (in bladder, head and neck, and non-small cell carcinoma); HPV EG and E7 proteins (in cervical cancer); Mucin [MUC-1](in breast, pancreas, colon, and prostate cancers); prostate-specific antigen [PSA](in prostate cancer); carcinoembryonic antigen [CEA](in colon, breast, and gastrointestinal cancers), and such shared tumor-specific antigens as MAGE-2, MAGE-4, MAGE-6, MAGE-10, MAGE-12, BAGE-1, CAGE-1,2,8, CAGE-3 TO 7, LAGE-1, NY-ESO-I/LAGE-2, NA-88, GnTV, TRP2-INT2, E6, E7, human glucagon receptor (hGCGR) and human ectonucleoside triphosphate diphosphohydrolase 3 (hENTPD3). Other targets can be selected by the person skilled in the art. See WO 2019/006046.

All numerical limits and ranges set forth herein include all numbers or values thereabout or there between of the numbers of the range or limit. The ranges and limits described herein expressly denominate and set forth all integers, decimals and fractional values defined and encompassed by the range or limit. Thus, a recitation of ranges of values herein are merely intended to serve as a way of referring individually to each separate value falling within the range, unless otherwise indicated herein, and each separate value is incorporated into the specification as if it were individually recited herein.

### DESCRIPTION

### Depth Filtration

Cell lysis is an early step in AAV purification. Detergents can be used to lyse cells to release proteins and viruses contained with the cell. Detergents to lyse cells are usually considered mild detergents and include: sodium dodecyl sulphate (SDS), NP-40, Tweens (for example 20 and 80), Tritons (for example X-100 and X-114), CHAPS, CHAPSO, Brij (for example, 35 and 58), Octyl thioglucoside, Octyl Glucoside, deoxycholate, and alkyl sulfates, for example.

Depth filtration typically follows lysis. Single-use chromatographic clarification uses a next-generation synthetic fibrous anion-exchange chromatographic clarification media to simultaneously conduct depth filtration, removal of cellular debris, soluble negatively charged impurities, and sterilize filter cell harvest material. Although traditionally used for mAb processing, these filters offer a unique benefit for rAAV production as they can process chromatin-containing material without excessive reduction in capacity, presenting an opportunity to eliminate expensive endonuclease (for example, Benzoase) treatment.

Harvest RC (3M) is a single-use chromatographic clarification unit consisting of synthetic fibrous anion exchange (AEX) media and a 0.2 μm polyethersulfone (PES) membrane. Cells are bound inside the fibrous AEX media by, allowing efficient retention of large and small particles without caking that rapidly fouls the filter. This structure also captures large strands of DNA without rapid fouling, unlike conventional depth filters which require the DNA to be digested by endonuclease prior to clarification.

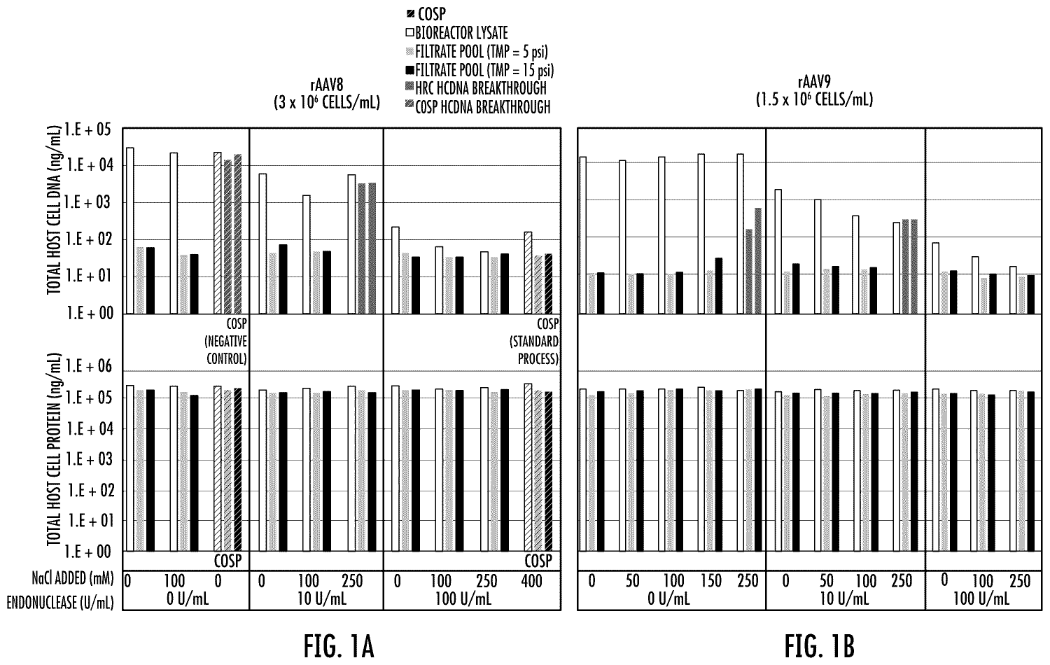

Tests with the Harvest RC (HRC) filters were conducted under different conditions of endonuclease addition and salt treatment. Comparison runs were also conducted with traditional COSP depth filtration media made of polypropylene fibers. HRC data and COSP data are set forth in FIG. 1A (rAAV8 at 3×10⁶ cells/ml) and FIG. 1B (rAAV9 at 1.5×10⁶ cells/ml). The top figures are for Total Host Cell DNA (ng/ml) and the bottom figures are for Total Host Cell Protein (ng/ml).

FIG. 1A is a bar graph for AAV8 depicting in order bars for bioreactor lysate (white bars), filtrate pool where transmembrane pressure (TMP) is 5 psi (light gray bars), and filtrate pool where TMP is 15 psi (black bars). HRC (solid bars) and COSP (a standard filter used as a control) (hatched and labeled bars) are filtration trains with different endonuclease and salt conditions in the load. FIG. 1B is a bar graph for AAV9 depicting in order bars for bioreactor lysate (white bars), filtrate pool where TMP is 5 psi (light gray bars), and filtrate pool where TMP is 15 psi (black bars). HRC (solid bars) are filtration trains with different endonuclease and salt conditions in the load. In FIG. 1A, significant breakthrough of HCDNA into the filtrate was observed for COSP at the “COSP Negative Control”, and for HRC at 250 mM NaCl and 0 U/ml endonuclease (dark gray bars). In FIG. 1B, significant breakthrough of HCDNA into the filtrate was observed for HRC at 250 mM NaCl and 0 U/ml endonuclease (dark gray bars) and 250 mM NaCl and 10 U/ml endonuclease (dark gray bars).

Notably, the COSP filter was unable to achieve significant removal of Host Cell DNA in the endonuclease-free train, with greater than 10⁴ ng/ml present in both the 5 and 15 psi fractions. In contrast, the HRC filters successfully removed Host Cell DNA to less than 10² ng/ml in all filtration trains in low-salt conditions for rAAV8 (FIG. 1A). In particular, for the endonuclease-free-trains a 100-fold reduction in host cell DNA (“HCDNA”) was achieved, from greater than 10⁴ ng/ml in the load material to less than 10² ng/ml in the filtrate fractions.

Additionally, capsid binding was not observed in any of the experiments on bioreactor harvest material, likely due the presence of sufficient HCDNA and other negatively charged impurities to compete with the capsids for the charged binding sites on the Harvest RC filter.

However, in the high salt condition when 250 mM NaCl was added to the load material, the HRC filter was unable to achieve effective Host Cell DNA (HCDNA) removal for the medium-endonuclease case, though the high salt level did not affect the filtrate quality in the high-endonuclease case. This is likely due to the salt competing with the Host Cell DNA for binding onto the anion exchange sites on the HRC filter, lowering the binding capacity. Finally, no significant reduction or impact on Host Cell Protein was observed between the load material and the filtrate fractions for any of the tested conditions.

For rAAV9, the low-salt and endonuclease-free conditions once again performed remarkably well, with 1000-fold reduction of Host Cell DNA from greater than 10⁴ ng/ml in the load material to 101 ng/ml in both the 5 and 15 psi filtrate fractions for 0-100 mM NaCl. See FIG. 1B. In all runs, it was observed that high salt conditions of 250 mM NaCl led to significant Host Cell DNA breakthrough into the filtrate for both the 0 U/mL and 10 U/mL endonuclease filtration trains, showing that limiting salt addition is critical for endonuclease-free clarification to be successful. The data with AAV8 and AAV9 show that an endonuclease-free clarification process can provide reduction of Host Cell DNA to comparable levels as conventional processes that use 100 U/mL of endonuclease in the bioreactor during lysis.

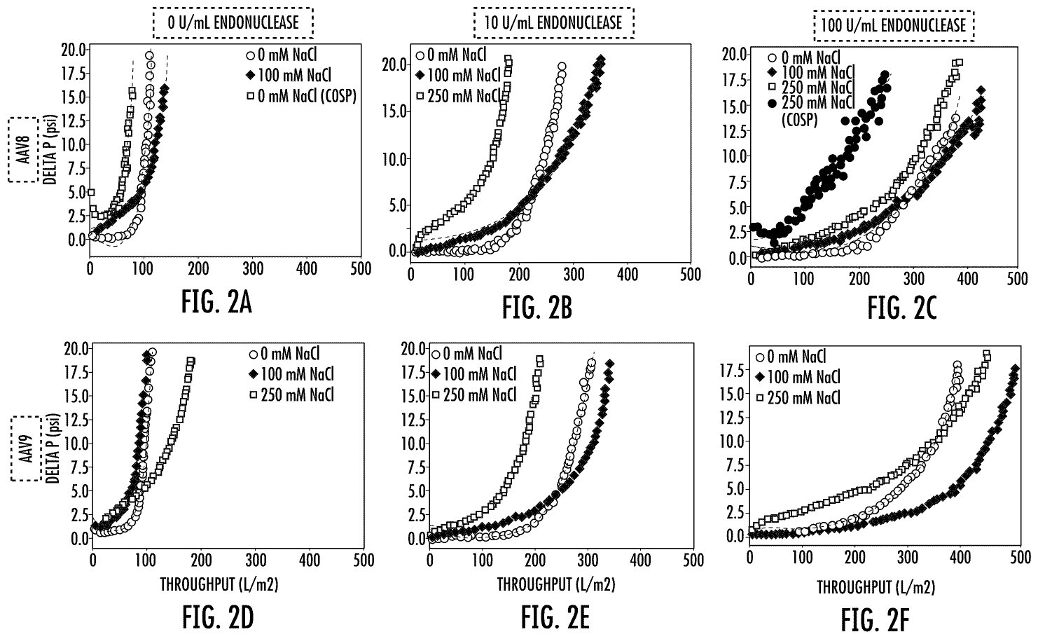

FIGS. 2A-2F provide data for depth filtration treatment at various salt and endonuclease conditions. FIGS. 2A-2C provide data from HRC depth filtration of AAV8 and FIGS. 2D-2F provide data from HRC depth filtration of AAV9. FIGS. 2A and 2D have no endonuclease added. FIGS. 2B and 2E have 10 units/ml of an endonuclease added. FIGS. 2C and 2F have 100 units/ml of an endonuclease added. Each of FIGS. 2B-2F contain data with concentrations of 0 mM NaCl, 100 mM NaCl and 250 mM NaCl. FIG. 2A contains data with concentrations of 0 mM NaCl and 100 mM NaCl with HRC and 0 mM NaCl with COSP. FIG. 2C data with concentrations of 0 mM NaCl, 100 mM NaCl and 250 mM NaCl for HRC and 250 mM NaCl for COSP.

Data is shown from HRC and COSP with AAV8 and AAV9 indicates Host Cell DNA breakthrough in FIG. 2A (0 mM NaCl with 0 U/m endonuclease) (COSP) (AAV8), FIG. 2B (250 mM NaCl with 10 U/m endonuclease) (HRC) (AAV8), FIG. 2D (250 mM NaCl with 0 U/m endonuclease) (HRC) (AAV9), and FIG. 2E (250 mM NaCl with 10 U/m endonuclease) (HRC) (AAV9). Experimental characterization of the effect of salt shows that it is important to limit salt addition to prevent Host Cell DNA breakthrough into the filtrate. However, an amount of salt (for example, less than 100 mM) can be beneficial to increase overall process throughput and prevent the differential pressure across the filter from increasing too steeply.

Overall, this approach enables endonuclease-free clarification of rAAV. Thus, using salt conditions of 0-100 mM, for example, clarification can be undertaken with low endonuclease or without an endonuclease altogether, and thereby significantly reduce purification costs.

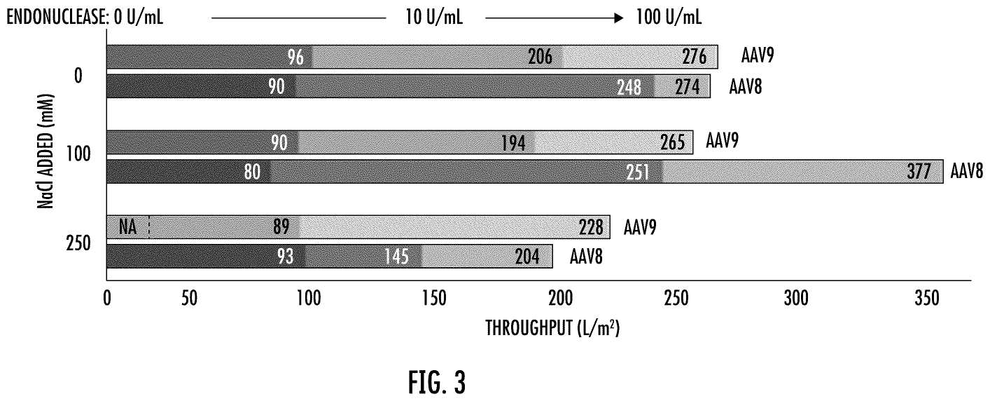

FIG. 3 depicts data set forth in FIGS. 2A-2F. A three-fold in throughput (liters filtered per meters² of filter area) at 5 pounds per square inch (psi) differential pressure at different endonuclease conditions, specifically at 0 U/ml, 10 U/ml and 100 U/ml of endonuclease, using AAV8 and AAV9. The 10 U/ml condition was midway between the 0 U/ml and 100 U/ml endonuclease conditions. Capsid and genomic yields in the filtrate pool of all runs were greater than 90% and comparable throughputs were observed at scales of 3.2 cm² and 25 cm².

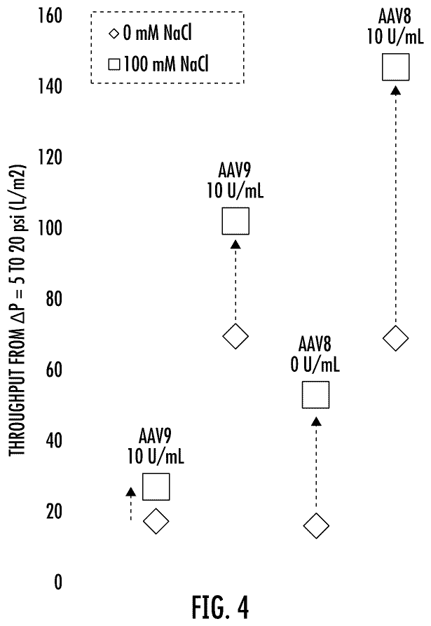

FIG. 4 depicts data set forth in FIGS. 2A-2F regarding the effects of salt (NaCl). The pressure range from 5-20 psi is a safe zone for flush and filter blowdown operations with depth filtration. If the pressure increases too sharply from 5-20 psi, there can be a loss of hold-up material because the modules should not be operated above a limit of 20 psi. Salt conditions, such as about 1 mM to 110 mM or more, but less than 250 mM. Preferably 1 mM to 100 mM, more preferably 1 mM to 75 mM, still more preferably 1 mM to 50 mM, and yet more preferably 1 mM to 25 mM. For example, preferred salt conditions such as 5 mM, 10 mM, 15 mM, 20 mM, 25 mM, 30 mM, 35 mM, 40 mM, 45 mM, 50 mM, 60 mM, 65 mM, 70 mM, 75 mM, 80 mM, 85 mM, 90 mM, 95 mM, 100 mM, 10⁵ mM, and 110 mM and ranges formed between any of these values (for example, 25 mM to 100 mM, 50 mM to 100 mM, 75 mM to 100 mM, 90 mM to 110 mM) can be selected.

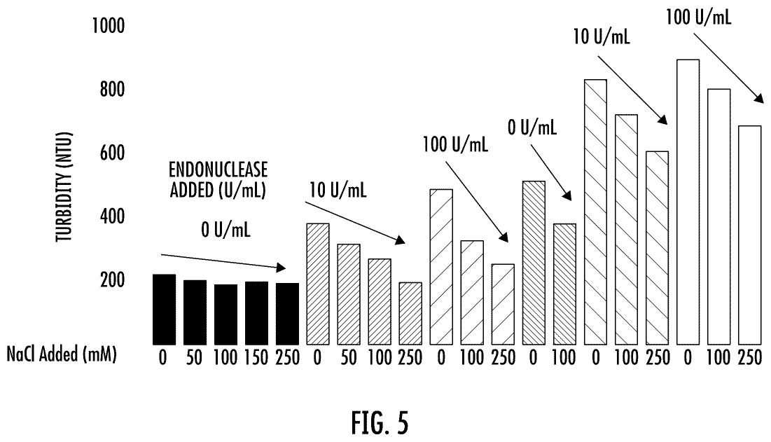

FIG. 5 depicts data showing that salt (NaCl) addition (See FIG. 4) reduces load turbidity and increases throughput at higher pressures. For example, salt conditions, such as about 1 mM to 110 mM or more, but less than 250 mM. Preferably 1 mM to 100 mM, more preferably 1 mM to 75 mM, still more preferably 1 mM to 50 mM, and yet more preferably 1 mM to 25 mM. For example, preferred salt conditions such as about 5 mM, 10 mM, 15 mM, 20 mM, 25 mM, 30 mM, 35 mM, 40 mM, 45 mM, 50 mM, 60 mM, 65 mM, 70 mM, 75 mM, 80 mM, 85 mM, 90 mM, 95 mM, 100 mM, 10⁵ mM, and 110 mM, and ranges formed between any of these values (for example, 25 mM to 100 mM, 50 mM to 100 mM, 75 mM to 100 mM, 90 mM to 110 mM) can be selected to reduce turbidity and increase throughput at higher pressures.

Treatment with 250 mM NaCl resulted in host cell DNA breakthrough into the filtrate for low and zero-endonuclease conditions.

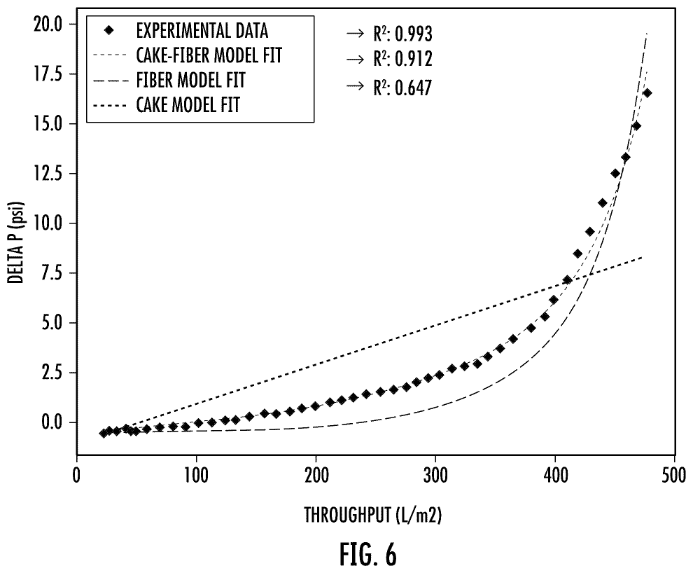

FIG. 6 depicts data set forth in FIGS. 2A-2F showing that a combined cake-fiber fouling model is a good representation for the mechanism of Harvest RC filtration (depth filtration). In FIG. 6, a filtration train with 0 U/ml endonuclease and 100 mM NaCl with AAV9 was analyzed.

Harvest RC filters contain fibrous anion exchange media layered above a flat sheet sterile filter. Low to zero endonuclease results in long strands of host cell DNA that are expected to bind to the AEX fibers, thereby causing fiber coating. Cake formation is expected at the flat-sheet filter and between tightly packed fibers. A cake-fiber fouling model resulted in excellent fit with R²>0.95 and was better than cake formation or fiber coating models individually.

The following model can be used:

$$\frac{Δ P}{Δ {P}_{0}} = \frac{[ 1 + \frac{( 1 - {∅}_{0} )}{{∅}_{0}} {K}_{f} V ]}{{( 1 - {K}_{f} V )}^{3}} + {K}_{c} {J}_{0} V$$

K_{c}=cake filtration constant. s/m²
K_{f}=fiber coating constant, 1/m
V=volume filtered, rn³/m²
P=pressure, kg/ms²
J=solvent flux, m/s
Ø=filter solid or fiber volume fraction

See G. R. Bolton, D. LaCasse, M. J. Lazzara, and R. Kuriyel, “The fiber-coating model of biopharmaceutical depth filtration,” AIChE Journal, vol. 51, no. 11. Wiley, pp. 2978-2987 (2005).

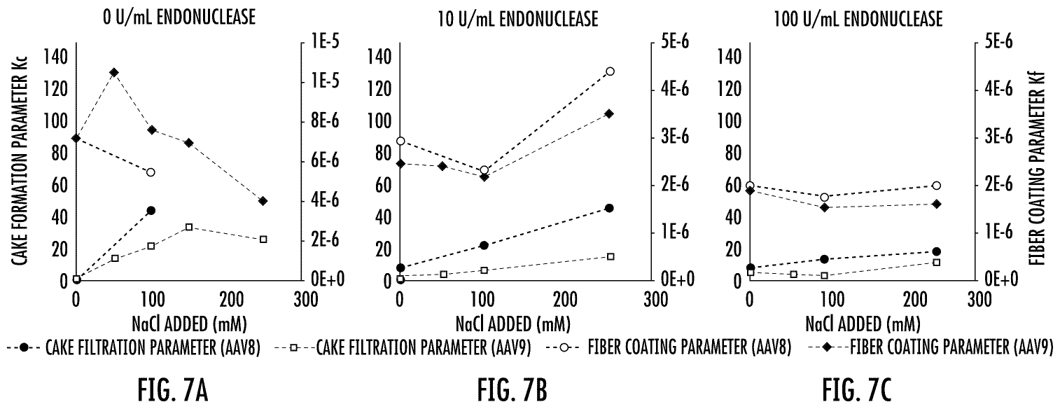

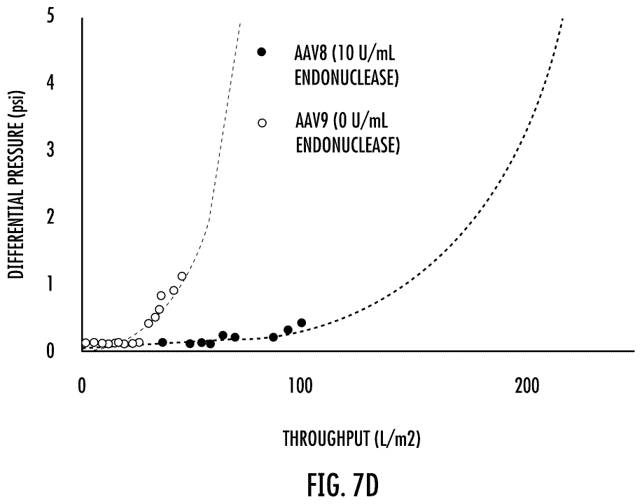

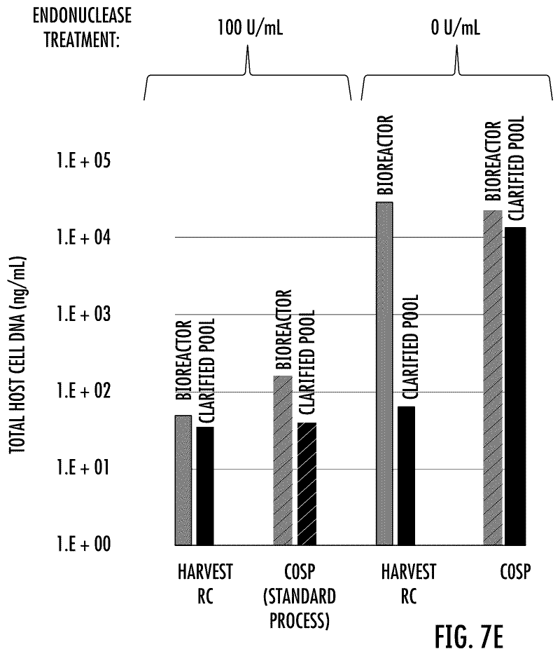

FIGS. 7A-7C depict data set forth in FIGS. 2A-2F. Cake formation parameters (K_{c}) vs NaCl (mM) added with 0 U/ml endonuclease (FIG. 7A), 10 U/ml endonuclease (FIG. 7B), and 100 U/ml endonuclease (FIG. 7C).

Empirical parameters extracted from the best-fit model curves were plotted for the different load conditions. The cake-fiber fouling model uses two parameters, namely cake formation parameter K_{c} and fiber coating parameter Kt. K_{c} increased with increasing salt for all endonuclease conditions. This is expected as CI-competes with HCDNA for AEX binding sites and causes more of a cake filtration than fiber coating mechanism. Kr decreased two-fold between the 0 U/mL and the 10 U/ml endonuclease conditions, from an average of 6×10⁻⁶ to 3×10⁻⁶. This suggests that the fiber coating effect is stronger in the endonuclease-free condition. That is, the long strands of undigested HCDNA are binding strongly onto the AEX fibers and creating a thickening effect.

The methods of the inventions provided a reduction of HCDNA from 5×10⁴ ng/ml to 50 ng/ml for rAAV8 and 2×10⁴ ng/mL to 10 ng/mL for rAAV9 in endonuclease-free conditions. This is comparable to traditional depth filtration media with 100 U/mL endonuclease treatment in the load.

FIG. 7D depicts filtration performance at 50 L scale that was evaluated with rAAV9 and rAAV8 See Table 1 below. Loads were pre-treated with 100 mM NaCl and either 10 U/mL and 0 U/mL endonuclease, respectively, and passed over two or five BC2300 filter units resulting in 100 L/m² or 50 L/m² filter loading. The filter areas were 30% oversized for the pilot-scale runs to provide margin for scale-up and enable smooth processing of the material. Differential pressure data for the filtration operations are shown in FIG. 7D illustrating below 2 psi maximal differential pressure.

In FIG. 7D, the model-predicted pressure profile up to 5 psi is overlaid in dashed lines, showing estimated capacity of about 200 L/m² for the rAAV8 trial and about 80 L/m² for the rAAV9 trial, consistent with the results from the bench-scale experiments with the comparable levels of endonuclease. Host cell DNA was reduced from 2×10⁵ ng/ml in the load of both serotypes to less than 50 ng/ml for rAAV8 and to less than 10 ng/ml for rAAV9, with a greater than 85% process yield in both trials, once again consistent with the bench-scale experiments.

TABLE 1

| AAV | | Endo- | | | |
|---|---|---|---|---|---|
| Serotype, | | nuclease | NaCl | Load | |
| Material | Filter | Added | Added | Conductivity | Load |
| Type | Type | (U/ml) | (mM) | (mS/cm) | pH |
| rAAV8, 50 L | HRC 2300 | 10 | 100 | 22 | 8.0 |
| Bioreactor | cm² Pod | | | | |
| Lysate | | | | | |
| rAAV9, 50 L | HRC 2300 | 0 | 100 | 22 | 8.0 |
| Bioreactor | cm² Pod | | | | |
| Lysate | | | | | |

Based on the 50 L trials, a preliminary cost-of-goods analysis at 500 L scale was carried out for the endonuclease-free process and reduced-endonuclease process (10 U/ml) in comparison to the standard 100 U/mL endonuclease condition. The cost-of-goods includes the cost of the required number of 1.6 m² BC16000 HRC filters needed process the 500 L batch estimated from the throughput achieved at 5 psi differential pressure. The reduced-endonuclease approach reduced overall cost about 83% and the endonuclease free approach reduced cost about 89%.

FIG. 7E is a summary chart comparing the performance of Harvest RC and COSP in the bioreactors and the clarified pool at endonuclease concentrations of 100U/ml or 0 U/ml. The use of chromatographic clarification according to the inventions can provide clarified pools with nearly the same level of host cell DNA with or without the use of endonuclease.

Overall, the methods of the inventions described herein permit endonuclease-free clarification of rAAV. Thus, using low salt conditions, clarification can be undertaken with low endonuclease or without an endonuclease altogether, and thereby significantly reduce purification costs.

Following depth filtration, the pool can be subjected to tangential flow filtration for concentration and buffer exchange before further chromatography, such as affinity chromatography.

### Affinity Chromatography

Purification of retargeted AAVs requires effective methods to capture surface-modified AAVs from the bioreactor harvest mixture. Challenges in affinity capture include difficulty in binding to the AAV surface epitopes in case of heavy conjugation, or difficulty in eluting the surface-modified AAV without destabilizing the molecule, thereby leading to aggregation, precipitation, and/or fragmentation. These difficulties often lead to an imbalance in the level of conjugation in the affinity capture load material relative to the eluate stream; that is, a loss of the some to all of the conjugated species during affinity capture due to preferential purification of the less conjugated and unconjugated AAVs. The goal of high-yield affinity capture of conjugated AAV species was not met until the present inventions.

Several screening runs were carried out on different resins and with different elution buffers. Poros CaptureSelect AAVX did not demonstrate good binding affinity for conjugated rAAV species, for example, AAV9-SpyT-SpyC-mAb. In contrast, Poros CaptureSelect AAV9 resin did exhibit good binding with conjugated rAAV species, for example, AAV9-SpyT-SpyC-mAb, but the yield was dependent on the choice of elution buffers.

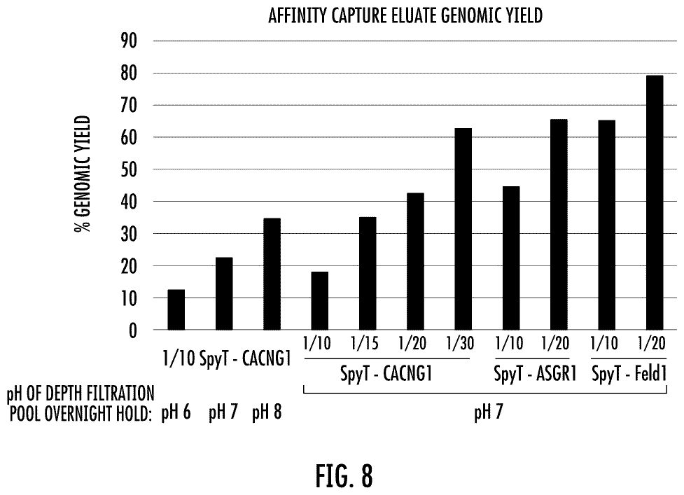

FIG. 8 depicts data using a POROS CaptureSelect AAV9 resin to capture AAV9-SpyT-SpyC-mAb. Monoclonal antibodies against CACNG1, ASGR1 and Fel d 1 were used as retargeting molecules. Average column loading was 2.5×10¹² viral genomes/ml resin and 9.2×10¹³ capsids/ml resin.

Less conjugated AAV9 (1/20 and 1/30) achieved higher yields. It is believed that affinity chromatography favored less conjugated AAV9 because such AAV9 would have more available epitopes.

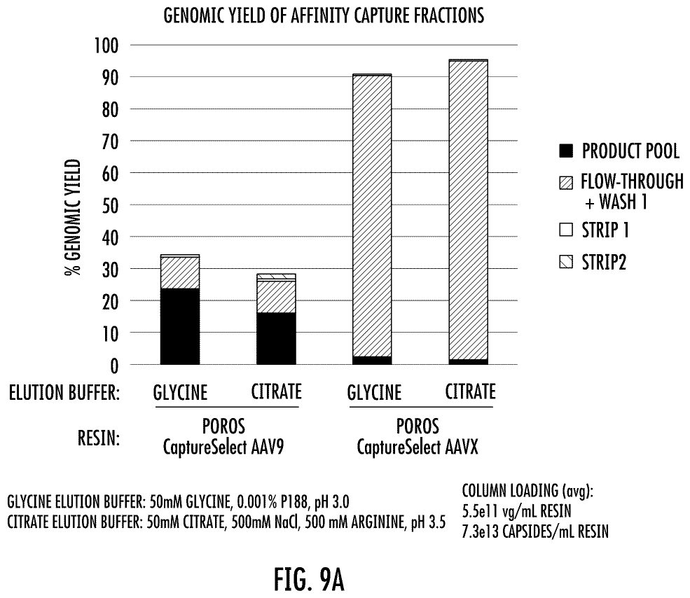

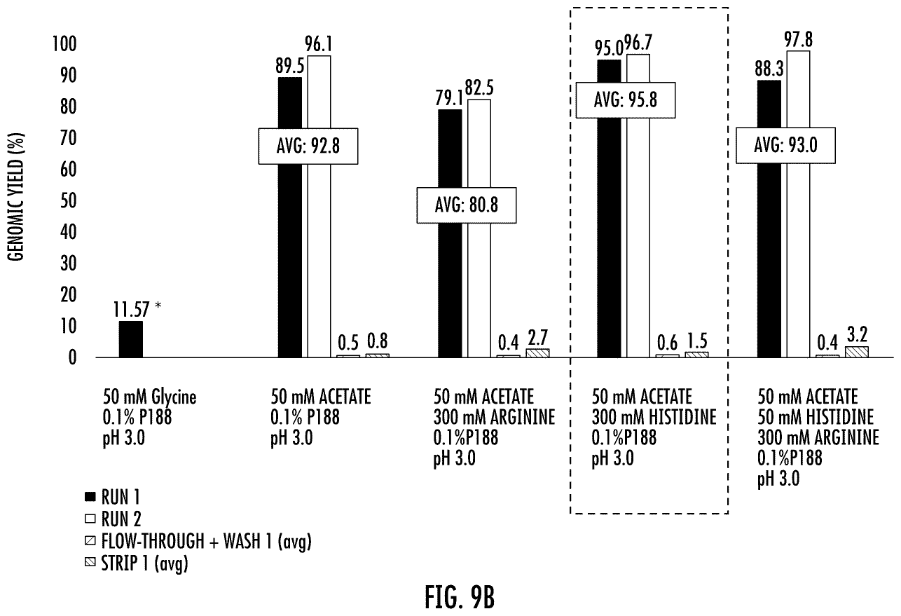

Columns and buffers were analyzed next. POROS CaptureSelect AAV9 and POROS CaptureSelect AAV X using citrate or glycine elution buffers were compared for their ability to capture conjugated AAV9. See FIG. 9A. It was hypothesized that during elution aggregation and degradation would lead to low yields of about 16-23%.

POROS CaptureSelect AAV9 using a glycine buffer proved superior for conjugated AAV9. Next, FIG. 9B compared various buffers used for affinity capture of an AAV-SpyT-SpyC-Tfr Fab. The buffers left to right comprised (i) glycine, (ii) acetate, (iii) acetate and arginine, (iv) acetate and histidine, and (v) acetate, histidine and arginine. Buffer (iv) acetate and histidine provided the highest yield. A POROS CaptureSelect AAV9 column loaded with 10¹³ to 10¹⁵ capsids per milliliter was employed.

Low yields were achieved in many elution buffers, such as yields of approximately 10% for glycine buffers along with loss of the heaviest conjugated species. A buffer comprising about: 50 mM acetate, 50 mM histidine, 0.001% P188, pH 3.0 was able to achieve yield of greater than 90% along with successful capture of the heavily conjugated AAV species. Notably, this buffer also resulted in highest yield for unconjugated AAV9-SpyTag species as produced in a post-processing conjugation system instead of a hexad transfection system.

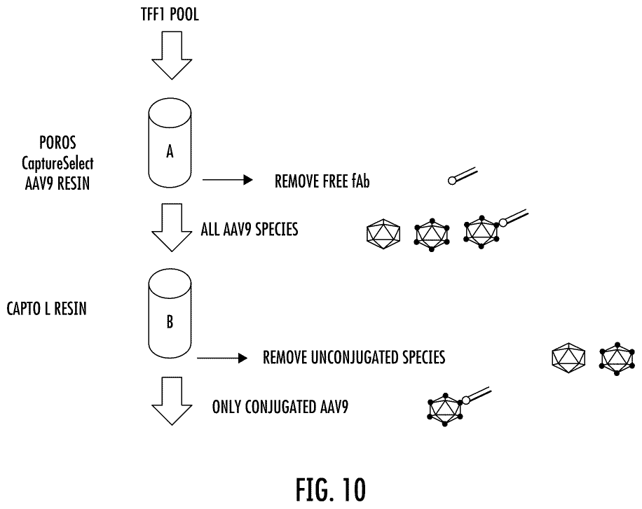

FIG. 10 compared the level of conjugation in the eluate of affinity capture using (i) a single affinity capture step specific to AAV surface epitopes, and (ii) after a second affinity capture step specific to antibody surface epitopes. POROS CaptureSelect AAV9 was useful for removing free antibody (fAb). The two-step affinity capture (POROS CaptureSelect AAV9 and Capto L) strategy resulted in removal of unconjugated AAV species. See FIG. 10 and Table 2.

TABLE 2

| | Fab-AAV9 | AAV9 Peak | Overall Yield |
|---|---|---|---|
| | Peak Area | Area | (droplet |
| Sample | (SEC-MALS | (SEC-MALS | digital PCR) |
| POROS CaptureSelect | 3950 | 3548 | 95% |
| AAV9 | | | |
| POROS CaptureSelect | 4025 | Not | 55% |
| AAV9 and Capto L | | detected | |

After depth filtration, and prior to or following affinity capture, the pool optionally can be subjected to tangential flow filtration (TFF) for concentration. Conventional TFF can be employed, which requires a batch approach and repeated cycling through the membrane to create a permeate and a retentate. Alternatively, single-pass TFF can be employed to allow for a continuous process. According to the inventions, TFF can advantageously employ permeate pumps to reduce TMP buildup and flux decline. Although not to be bound by any hypothesis or theory, it is believed that detergents, such as Tweens, build up on the retentate side of a TFF membrane and cause TMP buildup and flux decline. Single-Pass TFF units are available from Pall/Cytiva, Repligen and Millipore.

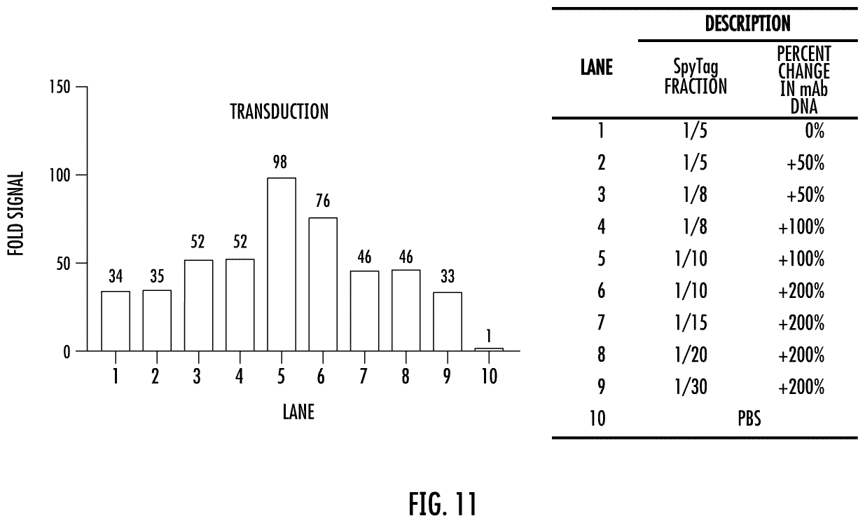

Affinity chromatographic purification was used for transduction assays. FIG. 11 is a bar graph depicting data relating to the selection of SpyTag fraction and SpyCatcher mAb DNA concentration. Spy Tag fractions ranged from 1/5 (pRC-SpyT to pRC is 1:4) to 1/30 (pRC-SpyT to pRC is 1:29). The percent change in mAb DNA concentration ranged 0% to 200%, meaning 0%=0.28 μg/ml, 100%=0.56 μg/ml, 200%=1.12 μg/ml. Lane 5, which had a 1/10 SpyTag fraction (pRC-Spy T to pRC is 1:9) and a 100% increase in mAb DNA showed the highest transduction efficiency. The signal was generated using a green fluorescent reporter gene.

### Multiple Pass Anion Exchange

To overcome purification challenges, the inventions also provide a multiple pass anion exchange (AEX) chromatography method which is effective in maximally enriching the pool for full capsids, as described herein. The use of AEX is often referred to as polishing, and typically would be undertaken following depth filtration and affinity capture of AAV.

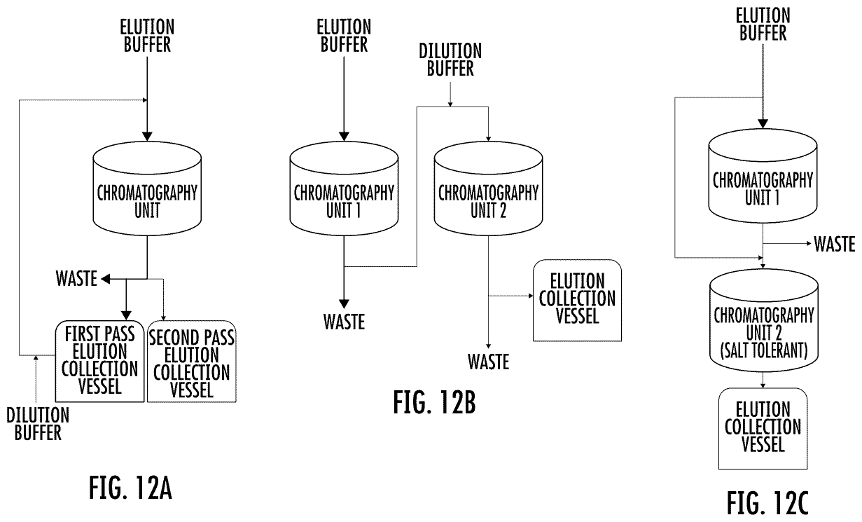

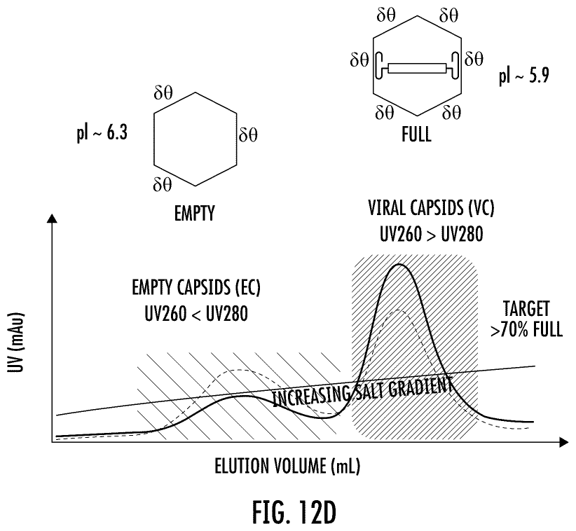

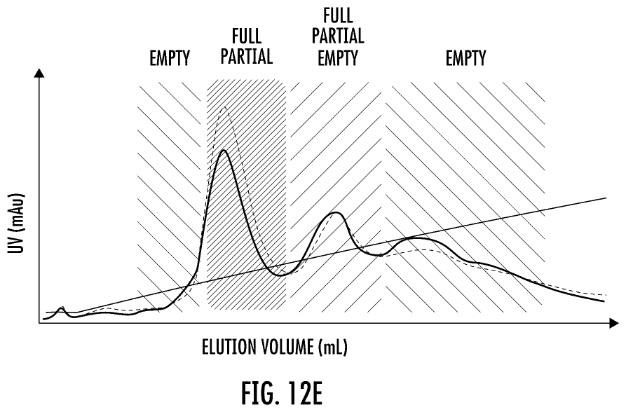

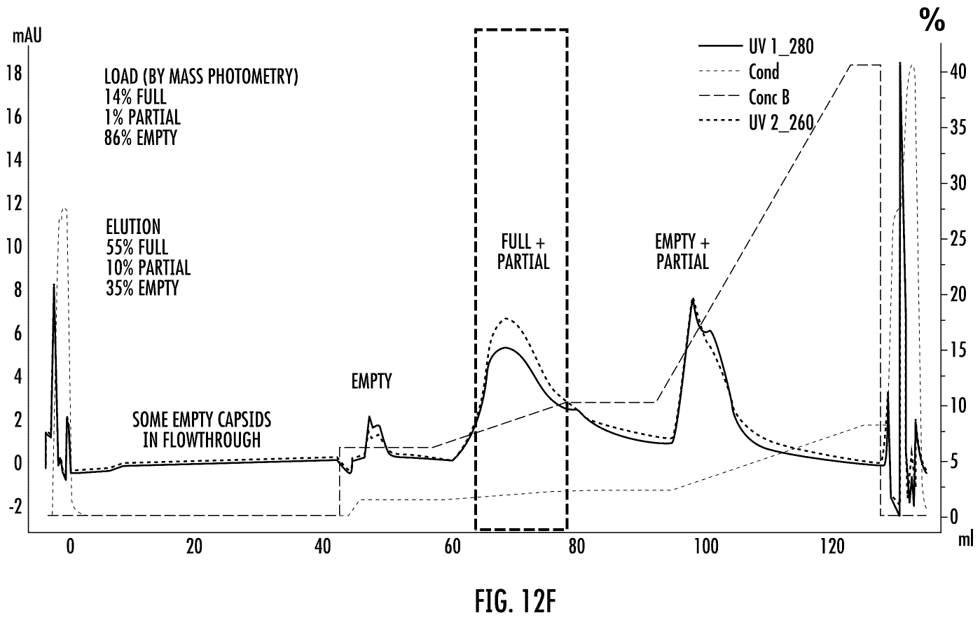

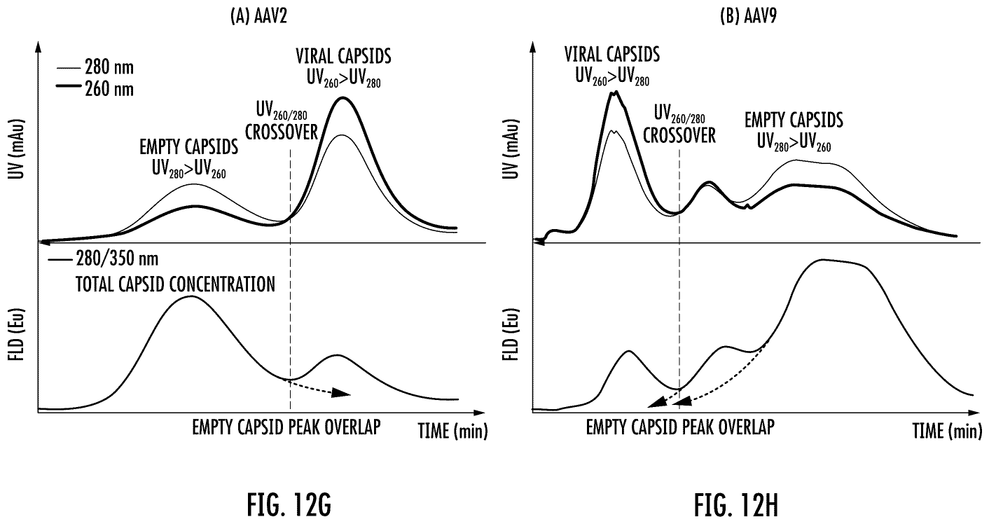

FIGS. 12A-12C describe different setups that can be used to carry out two-pass purification described in according to the inventions. FIG. 12A describes the setup for two or more passes on the same chromatography unit. FIG. 12B describes the setup for two passes on different chromatography units which can be used for continuous operation. FIG. 12C describes the setup for two passes with the second pass on a salt-tolerant chromatographic unit eliminating the need for in-line dilution.

There will exist empty and partially-filled capsid populations, some of which can have isolectric points (pl) that are above the pl of the full capsids, and others that can have isolectric points that are below the pl of the full capsids. FIG. 12D depicts a model elution of AAV (1, 2 and 8) empty capsids and full capsids using an increasing salt gradient. Empty capsids show greater absorbance at UV280 over UV260. Full capsids present the opposite—greater absorbance at UV260 over UV280. Empty capsids have a pl of about 6.3, whereas full capsids have a pl of about 5.9. The target would be about 70% full or greater.

FIG. 12E depicts exemplary elution profiles for AAV9 and AAV9-SpyT for empty, partially filled and full capsids, where a small population of empty capsids elutes before the full capsids, and a larger population of empty capsids elutes after the full capsids. These two profiles in FIGS. 12D and 12E serve as a model for other AAVs

FIG. 12F is a graph depicting separation empty, partially filled and full AAV9-SpyT capsids using a hybrid gradient combining linear and step gradients. The column was a CIM QA monolith loaded with 5×10¹³ to 5×10¹⁴ capsids (cp/ml). The buffer was 20 mM Bis-Tris-Propane, 0.001% P188, pH 9.6 and 1M NaCl and the process resulted in enrichment of full capsids from 14% to 55%. This hybrid gradient enabled improved resolution of the full capsids (central peak) from the empty capsid populations on either side. However, the hybrid gradient is operationally challenging at scale and also does not result in full capsids above the preferred threshold of 70%.

FIGS. 12G and 12H depict fluorescence emission intensity profiles (lower graphs) along with UV280 and UV260 profiles (upper graphs) for AAV2 (FIG. 12G) and AAV9 (FIG. 12H). The fluorescence intensity is a measure of true capsid concentration as it is unaffected by the presence or absence of DNA inside the capsids. The profiles show that the empty capsid peaks appear smaller than the full capsid peaks when measured by UV, but their true size is evident when using fluorescence, showing that empty capsids are more abundant than full capsids. The “overlap” region between the empty and full capsid peaks indicates suboptimal separation using linear gradients alone.

The inventions allow the downstream purification train to achieve full performance (high percentage of full capsids) even in the case of a low starting percentage of full capsids from the bioreactor, or when there is variability in the characteristics of the not full capsid population(s). The bulk of not full capsids are removed in the first pass. A stepwise elution of the first anion exchange treatment (first AEX pass) facilitates initial collection of full capsids and the subsequent anion exchange treatment (second, third, fourth or more AEX passes) allows for high enrichment, thus allowing a base process to be used for large-scale purification without method redevelopment. The inventions support rapid generation of material for preclinical and clinical trials and commercial production without extensive time spent on anion exchange process optimization for each new molecule or upstream process change.

The separation and purification methodologies of use a first and second AEX pass, or more, are amenable to all AAVs, including native AAVs, recombinant AAVs comprising a GOI and flanking AAV inverted terminal repeats (ITRs), and covalently surface modified AAV, including those that comprise GOls and flanking ITRs.

According to the inventions, the “first AEX pass” (step (a)) is the first time the AAV sample is loaded and eluted from the AEX chromatographic media, and the “second AEX pass” (step (b)) is the second time the AAV (from the pool of step (a)) is loaded and eluted from the AEX chromatographic media. An optional “third AEX pass” (step (c)) or further passes can be carried out. Elution is based upon a change in ionic strength achieved by mixing two elution buffers (Buffer A and Buffer B) with low and high ionic strength, respectively. The first pass can utilize a series of changes (“microsteps”) in ionic strength for elution, or gradient elution in which ionic strength is linearly increased from a starting to an ending value (microsteps are preferred). The second and subsequent passes can likewise utilize microsteps or gradient elution. The size of the change in ionic strength in each microstep can be determined from the range of ionic strength in which the full capsids elute in a linear gradient, by dividing the range into parts, such as 2, 3, 4, 5, 6, 7, 8, 9, or 10 parts (2 to 4 parts is preferred). For example, if the full capsids elute in an ionic strength range of 9 mS/cm to 15 mS/cm, corresponding to a difference of 6 mS/cm between the start and end of the range, the size of the microsteps can be 0.6 mS/cm to 3 mS/cm by dividing the range into 2 to 10 parts. The change in ionic strength can be referred to as a change in the percentage of Buffer B mixed with Buffer A. For example, if Buffer A has ionic strength of 1 mS/cm and Buffer B has ionic strength of 20 mS/cm, a microstep of 0.6 mS/cm will correspond to a 3.1% change in Buffer B, while a microstep of 3 mS/cm will correspond to a 15.8% change in Buffer B. Preferably, 2 to 4 parts is used because 1 part may be insufficient for partitioning between full and capsids that are not full, while more than 4 parts may lead to excessive diffusion and reduce resolution between elution peaks. Selecting the elution step size based on the full peak conductivity elution range in a linear gradient allows a consistent approach to be used across different serotypes and modalities with different elution conductivity ranges. Table 3 below described the selection of ionic strength for the microstep elution for different AAV types using f=3 where f is the number of parts into which the linear elution range is divided to convert the linear gradient elution into microsteps.

TABLE 3

| | Conductivity | | | | | |
|---|---|---|---|---|---|---|
| | range of full | | | | | |
| | peak in linear | | Buffer A | Buffer B | Calculated | Calculated |
| AAV and AEX | gradient | | conductivity | conductivity | step size | step size |
| column | (mS/cm) | f | (mS/cm) | (mS/cm) | (mS/cm) | (% B) |
| AAV2 | 7.3 to 9.0 | 3 | 0.5-1.0 | 18.5 | 0.6 | 3.1 |
| (CIM QA) | | | | | | |
| AAV8 | 11.0 to 12.5 | 3 | 0.5-1.0 | 25.5 | 0.6 | 2.4 |
| (CIM QA) | | | | | | |
| AAV9 | 9.1 to 12.5 | 3 | 0.5-1.0 | 85.5 | 1.1 | 1.3 |
| (CIM QA) | | | | | | |
| AAV9-Spy | 9.0 to 14.0 | 3 | 0.5-1.0 | 85.5 | 1.7 | 2.0 |
| Tag | | | | | | |
| (CIM QA) | | | | | | |
| AAV9-Spy | 1.5 to 3.1 | 3 | 0.5-1.0 | 9.76 | 0.5 | 6.0 |
| Tag(Prima T) | | | | | | |
| AAV9-Spy | 1.5 to 4.0 | 3 | 0.5-1.0 | 21.4 | 0.8 | 4.0 |
| Tag-Spy | | | | | | |
| Catcher- | | | | | | |
| ASGR1 | | | | | | |
| mAb/-Tfr Fab | | | | | | |
| (Prima T) | | | | | | |

The AAV sample to be enriched typically will comprise less than 1%, 2%, 3%, 4%, 5% 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% full AAV capsids. Typically, the presence of full AAV capsids is 5% to 20% in the AAV sample to be enriched.

Enrichments of 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11 or more fold recovery of full AAV capsids are achievable. Enrichments of 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11 or more fold recovery of full plus partially filled capsids also are achievable.

The inventions are well-suited to complex separations in which there are multiple not full capsid populations with pl both above and below the pl of the full capsids, which facilitates fractionation of the full capsid peak in the second pass as the bulk of not full capsids are removed in the first pass.

### Cell Culture

The present inventions are amenable for purification of AAV produced in cell culture, such as mammalian cell culture. Exemplary mammalian cell lines are CHO, Per.C6 cells, Sp2/0 cells, and HEK293 cells. CHO cells include, but are not limited to, CHO-ori, CHO-K1, CHO-s, CHO-DHB11, CHO-DXB11, CHO-K1SV, and mutants and variants thereof. HEK293 cells include, but are not limited, to HEK293, HEK293A, HEK293E, HEK293F, HEK293FT, HEK293FTM, HEK293H, HEK293MSR, HEK293S, HEK293SG, HEK293SGGD, HEK293T and mutants and variants thereof. Adherent HEK 293 cells also can be used for production of covalently surface modified AAV according to the inventions. HEK 293 suspension cultured cells were derived from HEK 293 adherent cells. See Malm et al., Scientific Reports 10:18996 (2020). Other suitable cells include, but are not limited to BHK (baby hamster kidney) cells, Hela cells and Human Amniotic cells, such as Human Amniotic Epithelial cells. Other cell types for production include insect cells, such as Sf9.

### AAV and Recombinant AAV

Recombinant AAV of any serotype, for example, AAV1, AAV2, AAV2quad (Y-F), AAV3, AAV4, AAV5, AAV6, AAV7, AAV8, AAV9, AAV10, AAV11, AAV12, AAV13, rh10, rh39, rh43, rh74 Avian AAV, Sea Lion AAV, Bearded Dragon AAV, as well as variants thereof, can be modified with genes of interest using methodologies, approaches, genes and cells described in WO 2023/069931; WO 2023/069929; and WO 2023/069926.

### Covalently Surface Modified Recombinant AAV

Recombinant AAV capsids with modified viral capsid proteins to permit retargeting of AAV are disclosed in WO 2019/006046.

The modified AAVs described herein may be derived using a capsid gene of a non-enveloped virus. The modification can encoded by a cap gene modified to express a genetically modified capsid protein of a non-enveloped virus, wherein the non-enveloped virus infects human cells, or serotypes of non-enveloped viruses that generally infect human cells, such as adenovirus, adeno-associated virus, and others. A recombinant viral capsid protein described herein can derived from an AAV capsid gene that encodes the VP1, VP2, and/or VP3 capsid proteins of the AAV (or portions of the VP1, VP2, and/or VP3 capsid proteins). The modification can be encoded by a cap gene modified to encode a genetically modified adeno-associated virus (AAV) VP1, VP2 and/or VP3 capsid protein. These include genetically modified capsid proteins of an AAV serotype, for example, AAV1, AAV2, AAV2quad (Y-F), AAV3, AAV4, AAV5, AAV6, AAV7, AAV8, AAV9, AAV10, AAV11, AAV12, AAV13, rh10, rh39, rh43, rh74 Avian AAV, Sea Lion AAV, Bearded Dragon AAV, as well as variants thereof, and any variants thereof.

The inventions herein facilitate purification of full capsids having modified capsid proteins. Specifically, such modified capsid protein approaches utilize a first member and a second cognate member of a specific binding pair, which first member and second cognate member specifically interact to form a chemical, preferably covalent, bond. The first member, when displayed on a capsid protein, acts as a scaffold for any targeting ligand fused to the second cognate member, but upon binding of the first member and second cognate member, an isopeptide bond forms, and the recombinant viral particle acts as a one-component targeting vector. The covalently surface modified AAVs also can comprise GOIs and ITRs. See Example 5.

The second member can be operably linked to a targeting ligand, optionally wherein the targeting ligand is a binding moiety. The first member can be flanked by a first and/or second linker that link(s) the first member to the capsid protein, and wherein the first and/or second linker is each independently at least one amino acid in length. The first and second linker can be identical or non-identical.

Systems to facilitate retargeting include the Spy Tag: Spy Catcher system is described in U.S. Pat. No. 9,547,003 and Zakeri et al. (2012) PNAS 109: E690-E697, is derived from the CnaB2 domain of the Streptococcus pyogenes fibronecting-binding protein FbaB. See WO 2019/006046.

SpyTag002: SpyCatcher002 system is described in Keeble et al (2017) Angew Chem Int Ed Engl 56:16521-25. See WO 2019/006046.

SpyTag003: Spay Catcher003 also has been created. Spy Tag002: SpyCatcher002 and SpyTag003: SpyCatcher003 are different iterations of Spy Tag: Spy Catcher.

The SnoopTag: SnoopCatcher system is described in Veggiani (2016) PNAS 113:1202-07. The D4 Ig-like domain of RrgA, an adhesion from Streptococcus pneumoniae, was split to form SnoopTag. Incubation of SnoopTag and SnoopCatcher results in a spontaneous isopeptide bond that is specific between the complementary proteins. Veggiani (2016)), supra. See WO 2019/006046.

The Isopeptag: Pilin-C specific binding pair was derived from the major pilin protein Spy0128 from Streptococcus pyogenes. (Zakeir and Howarth (2010) J. Am. Chem. Soc. 132:4526-27). See WO 2019/006046.

Other systems to facilitate retargeting can be based upon the splitting and engineering of RegA domain 4. These have led to SnoopTagJr: SnoopCatcher, DogTag: DogCatcher and Snoop Ligase. Other systems include Isopeptag: Pilin-N, SdyTg: SdyCatcher, Jo: In, 3kptTag: 3kptCatcher, 40q1 Taq/4oq1 Catcher, NGTag/Catcher, Rumtrunk/Mooncake, GalacTag, Cpe, Ececo, Corio and all others based upon isopeptide binding pairs.

Due to the common properties, structures, and genomic sequences and organizations of AAVs, the present inventions are amenable to the enrichment of AAV, recombinant AAVs, and covalently surface modified recombinant AAVs of all serotypes, as well as variants thereof.

All major antibody classes, namely IgG, IgA, IgM, IgD and IgE, can be used as targeting molecules. IgG is a preferred class, and includes subclasses IgG1 (including IgG1) and IgG1K), IgG2, IgG3, and IgG4. Further antibody types include a human antibody, a humanized antibody, a chimeric antibody, a monoclonal antibody, a multispecific antibody, a bispecific antibody, a trispecific antibody, an antigen binding antibody fragment, a single chain antibody, a diabody, triabody or tetrabody, a Fab fragment or a F (ab′) 2 fragment, an IgD antibody, an IgE antibody, an IgM antibody, an IgG antibody, an IgG1 antibody, an IgG2 antibody, an IgG3 antibody, or an IgG4 antibody. Derivatives, components, domains, chains and fragments of the above also are included as types of targeting molecules.

As stated above, different AAV serotypes are known to preferentially transduce the cells of different tissues. Tissue specificity is limited, and AAV is known to preferentially transduce the liver, which can be a safety and efficacy concern in some contexts.

The inventions are further described by the following Examples, which do not limit the inventions in any manner and are applicable to all sections of the descriptions of the inventions and the aspects of the inventions. The order of performance of the below examples can be altered or combined as determined by the person of skill in the art in view of the teachings and data contained herein.

### Example 1—Depth Filtration with and without Endonuclease Under Varying Salt Conditions

Tests with the Harvest RC (HRC) filters were conducted under different conditions of endonuclease addition and salt treatment. Comparison runs were also conducted with traditional COSP depth filtration media made of polypropylene fibers. HRC data and COSP data are set forth in FIG. 1A (rAAV8) and FIG. 1B (rAAV9). The top figures are for Total Host Cell DNA (ng/ml) and the bottom figures are for Total Host Cell Protein (ng/ml).

Notably, the COSP filter was unable to achieve significant removal of Host Cell DNA in the endonuclease-free train, with greater than 10⁴ ng/ml present in both the 5 and 15 psi fractions. In contrast, the HRC filters successfully removed Host Cell DNA to less than 10² ng/mL in all filtration trains in low-salt conditions for rAAV8 (FIG. 1A). In particular, for the endonuclease-free-trains a 100-fold reduction in HCDNA was achieved, from greater than 10⁴ ng/ml in the load material to less than 10² ng/ml in the filtrate fractions.

However, in the high salt condition when 250 mM NaCl was added to the load material, the HRC filter was unable to achieve effective Host Cell DNA removal for the medium-endonuclease case, though the high salt level did not affect the filtrate quality in the high-endonuclease case. This is likely due to the salt competing with the Host Cell DNA for binding onto the anion exchange sites on the HRC filter, lowering the binding capacity. Finally, no significant reduction or impact on Host Cell Protein was observed between the load material and the filtrate fractions for any of the tested conditions.

For rAAV9, the low-salt and endonuclease-free conditions once again performed remarkably well, with 1000-fold reduction of Host Cell DNA from greater than 10⁴ ng/ml in the load material to 101 ng/ml in both the 5 and 15 psi filtrate fractions for 0-100 mM NaCl. See FIG. 1B. In all runs, it was observed that high salt conditions of 250 mM NaCl led to significant Host Cell DNA breakthrough into the filtrate for both the 0 U/mL and 10 U/mL endonuclease filtration trains, showing that limiting salt addition is critical for endonuclease-free clarification to be successful.

The data with AAV8 and AAV9 show that an endonuclease-free clarification process can provide reduction of Host Cell DNA to comparable levels as conventional processes that use 100 U/mL of endonuclease in the bioreactor during lysis.

### Example 2—Additional Depth Filtration Treatments at Different Salt and Endonuclease Conditions

FIGS. 2A-2F provide data for depth filtration treatment at various salt and endonuclease conditions. FIGS. 2A-2C provide data from HRC depth filtration of AAV8 and FIGS. 2D-2F provide data from HRC depth filtration of AAV9. FIGS. 2A and 2D have no endonuclease added. FIGS. 2B and 2E have 10 units/ml of an endonuclease added. FIGS. 2C and 2F have 100 units/ml of an endonuclease added. Each of FIGS. 2B-2F contain data with concentrations of 0 mM NaCl, 100 mM NaCl and 250 mM NaCl. FIG. 2A contains data with concentrations of 0 mM NaCl and 100 mM NaCl with HRC and 0 mM NaCl with COSP. FIG. 2C data with concentrations of 0 mM NaCl, 100 mM NaCl and 250 mM NaCl for HRC and 250 mM NaCl for COSP.

Data is shown from HRC and COSP with AAV8 and AAV9 indicates Host Cell DNA breakthrough in FIG. 2A (0 mM NaCl with 0 U/m endonuclease) (COSP) (AAV8), FIG. 2B (250 mM NaCl with 10 U/m endonuclease) (HRC) (AAV8), FIG. 2D (250 mM NaCl with 0 U/m endonuclease) (HRC) (AAV9), and FIG. 2E (250 mM NaCl with 10 U/m endonuclease) (HRC) (AAV9). Experimental characterization of the effect of salt shows that it is important to limit salt addition to prevent Host Cell DNA breakthrough into the filtrate. However, an amount of salt (for example, less than 100 mM) can be beneficial to increase overall process throughput and prevent the differential pressure across the filter from increasing too steeply.

Overall, this approach enables endonuclease-free clarification of rAAV. Thus, using salt conditions of 0-100 mM, for example, clarification can be undertaken with low endonuclease or without an endonuclease altogether, and thereby significantly reduce purification costs.

The results in Example 2 are consistent with the results in Example 1.

### Example 3—Additional Depth Filtration Analysis

FIG. 3 depicts data set forth in FIGS. 2A-2F. A three-fold in throughput (liters filtered per meters² of filter area) at 5 pounds per square inch (psi) differential pressure at different endonuclease conditions, specifically at 0 U/ml, 10 U/ml and 100 U/ml of endonuclease, using AAV8 and AAV9. The 10 U/ml condition was midway between the 0 U/ml and 100 U/ml endonuclease conditions. Capsid and genomic yields in the filtrate pool of all runs were greater than 90% and comparable throughputs were observed at scales of 3.2 cm² and 25 cm².

FIG. 4 depicts data set forth in FIGS. 2A-2F regarding the effects of salt (NaCl). The pressure range from 5-20 psi is a safe zone for flush and filter blowdown operations with depth filtration. If the pressure increases too sharply from 5-20 psi, there can be a loss of hold-up material because the modules should not be operated above a limit of 20 psi. Salt conditions, such as about 1 mM to 110 mM or more, but less than 250 mM. Preferably 1 mM to 100 mM, more preferably 1 mM to 75 mM, still more preferably 1 mM to 50 mM, and yet more preferably 1 mM to 25 mM. For example, salt conditions such as 5 mM, 10 mM, 15 mM, 20 mM, 25 mM, 30 mM, 35 mM, 40 mM, 45 mM, 50 mM, 60 mM, 65 mM, 70 mM, 75 mM, 80 mM, 85 mM, 90 mM, 95 mM, 100 mM, 10⁵ mM, and 110 mM, and ranges formed between any of these values, can be selected. The salt can be an organic or inorganic salt. Inorganic salts include sodium chloride, potassium chloride, magnesium chloride, calcium chloride, sodium bicarbonate, calcium carbonate, sodium sulfate, calcium phosphate, ammonium chloride, and ammonium sulfate.

FIG. 5 depicts data showing that salt (NaCl) addition (See FIG. 4) reduces load turbidity and increases throughput at higher pressures. For example, salt conditions, such as about 1 mM to 110 mM or more, but less than 250 mM. Preferably 1 mM to 100 mM, more preferably 1 mM to 75 mM, still more preferably 1 mM to 50 mM, and yet more preferably 1 mM to 25 mM. For example, preferred salt conditions such as 5 mM, 10 mM, 15 mM, 20 mM, 25 mM, 30 mM, 35 mM, 40 mM, 45 mM, 50 mM, 60 mM, 65 mM, 70 mM, 75 mM, 80 mM, 85 mM, 90 mM, 95 mM, 100 mM, 10⁵ mM, and 110 mM, and ranges formed between any of these values (for example, 25 mM to 100 mM, 50 mM to 100 mM, 75 mM to 100 mM, 90 mM to 110 mM) can be selected to reduce turbidity and increase throughput at higher pressures. The salt can be an organic or inorganic salt. Inorganic salts include sodium chloride, potassium chloride, magnesium chloride, calcium chloride, sodium bicarbonate, calcium carbonate, sodium sulfate, calcium phosphate, ammonium chloride, and ammonium sulfate.

Treatment with 250 mM NaCl resulted in host cell DNA breakthrough into the filtrate for low and zero-endonuclease conditions.

FIG. 6 depicts data set forth in FIGS. 2A-2F showing that a combined cake-fiber fouling model is a good representation for the mechanism of Harvest RC filtration (depth filtration). In FIG. 6, a filtration train with 0 U/ml endonuclease and 100 mM NaCl with AAV9 was analyzed.

Harvest RC filters contain fibrous anion exchange media layered above a flat sheet sterile filter. Low/zero endonuclease results in long strands of host cell DNA that are expected to bind to the AEX fibers, thereby causing fiber coating. Cake formation is expected at the flat-sheet filter and between tightly packed fibers. A cake-fiber fouling model resulted in excellent fit with R²>0.95 and was better than cake formation or fiber coating models individually.

The following model can be used:

$$\frac{Δ P}{Δ {P}_{0}} = \frac{[ 1 + \frac{( 1 - {∅}_{0} )}{{∅}_{0}} {K}_{f} V ]}{{( 1 - {K}_{f} V )}^{3}} + {K}_{c} {J}_{0} V$$

K_{c}=cake filtration constant. s/m²
K_{f}=fiber coating constant, 1/m
V=volume filtered, rn³/m²
P=pressure, kg/ms²
J=solvent flux, m/s
Ø=filter solid or fiber volume fraction

See G. R. Bolton, D. LaCasse, M. J. Lazzara, and R. Kuriyel, “The fiber-coating model of biopharmaceutical depth filtration,” AIChE Journal, vol. 51, no. 11. Wiley, pp. 2978-2987 (2005).

FIGS. 7A-7C depict data set forth in FIGS. 2A-2F. Cake formation parameters (K_{c}) vs NaCl (mM) added with 0 U/ml endonuclease (FIG. 7A), 10 U/ml endonuclease (FIG. 7B), and 100 U/ml endonuclease (FIG. 7C).

Empirical parameters extracted from the best-fit model curves were plotted for the different load conditions. The cake-fiber fouling model uses two parameters, namely cake formation parameter K_{c} and fiber coating parameter K_{f}. K_{c} increased with increasing salt for all endonuclease conditions. This is expected as Cl⁻ competes with HCDNA for AEX binding sites and causes more of a cake filtration than fiber coating mechanism. Kr decreased two-fold between the 0 U/mL and the 10 U/ml endonuclease conditions, from an average of 6×10⁻⁶ to 3×10⁻⁶. This suggests that the fiber coating effect is stronger in the endonuclease-free condition. That is, the long strands of undigested HCDNA are binding strongly onto the AEX fibers and creating a thickening effect.

The methods of the inventions provided a reduction of HCDNA from 5×10⁴ ng/ml to 50 ng/ml for rAAV8 and 2×10⁴ ng/mL to 10 ng/ml for rAAV9 in endonuclease-free conditions. This is comparable to traditional depth filtration media with 100 U/mL endonuclease treatment in the load.

Overall, the methods of the inventions described herein permit endonuclease-free clarification of rAAV. Thus, using low salt conditions, clarification can be undertaken with low endonuclease or without an endonuclease altogether, and thereby significantly reduce purification costs.

Following depth filtration, the pool can be subjected to tangential flow filtration for concentration and buffer exchange before further chromatography, such as affinity chromatography.

### Example 4—Affinity Chromatography to Purify Covalently Surface Modified AAV

FIG. 8 depicts data using a POROS CaptureSelect AAV9 resin to capture AAV9-SpyT-SpyC-mAb. Monoclonal antibodies against CACNG1, ASGR1 and Fel d 1 were used as retargeting molecules. Average column loading was 2.5×10¹² viral genomes/ml resin and 9.2×10¹³ capsids/ml resin.

Less conjugated AAV9 (1/20 and 1/30) achieved higher yields. It is believed that affinity chromatography favored less conjugated AAV9 because such AAV9 would have more available epitopes.

Columns and buffers were analyzed next. POROS CaptureSelect AAV9 and POROS CaptureSelect AAV X using citrate or glycine elution buffers were compared for their ability to capture conjugated AAV9. See FIG. 9A. It was hypothesized that during elution aggregation and degradation would lead to low yields of about 16-23%.

POROS CaptureSelect AAV9 using a glycine buffer proved superior for conjugated AAV9. Next, FIG. 9B compared various buffers used for affinity capture of an AAV-SpyT-SpyC-Tfr Fab. The buffers left to right comprised (i) glycine, (ii) acetate, (iii) acetate and arginine, (iv) acetate and histidine, and (v) acetate, histidine and arginine. Buffer (iv) acetate and histidine provided the highest yield at an average of 95.8%. A POROS CaptureSelect AAV9 column loaded with 10¹³ to 10¹⁵ capsids per milliliter was employed.

FIG. 10 compared the level of conjugation in the eluate of affinity capture using (i) a single affinity capture step specific to AAV surface epitopes, and (ii) after a second affinity capture step specific to antibody surface epitopes. POROS CaptureSelect AAV9 was useful for removing free antibody (fAb).

The two-step affinity capture (POROS CaptureSelect AAV9 and Capto L) strategy resulted in removal of unconjugated AAV species. See FIG. 10 and Table 2.

### Example 5—Affinity Chromatography in Transduction Assays

Affinity chromatographic purification was used for transduction assays. FIG. 11 is a bar graph depicting data relating to the selection of SpyTag fraction and SpyCatcher mAb DNA concentration. Spy Tag fractions ranged from 1/5 (pRC-SpyT to pRC is 1:4) to 1/30 (pRC-SpyT to pRC is 1:29). The percent change in mAb DNA concentration ranged 0% to 200%, meaning 0%=0.28 μg/ml, 100%=0.56 μg/ml, 200%=1.12 μg/ml. Lane 5, which had a 1/10 SpyTag fraction (pRC-Spy T to pRC is 1:9) and a 100% increase in mAb DNA showed the highest transduction efficiency. The signal was generated using a green fluorescent reporter gene.

This example shows that the middle conditions in FIG. 11 (1/10 SpyTag fraction and 0.56 μg/ml mAb concentration) were optimal for transduction.

### Example 6—Comparison of AEX Approaches with Recombinant AAV9

Different setups that can be used to carry out two-pass anion exchange purification described of these inventions. FIG. 12A describes the setup for two or more passes on the same chromatography unit. FIG. 12B describes the setup for two passes on different chromatography units which can be used for continuous operation. FIG. 12C describes the setup for two passes with the second pass on a salt-tolerant chromatographic unit eliminating the need for in-line dilution.

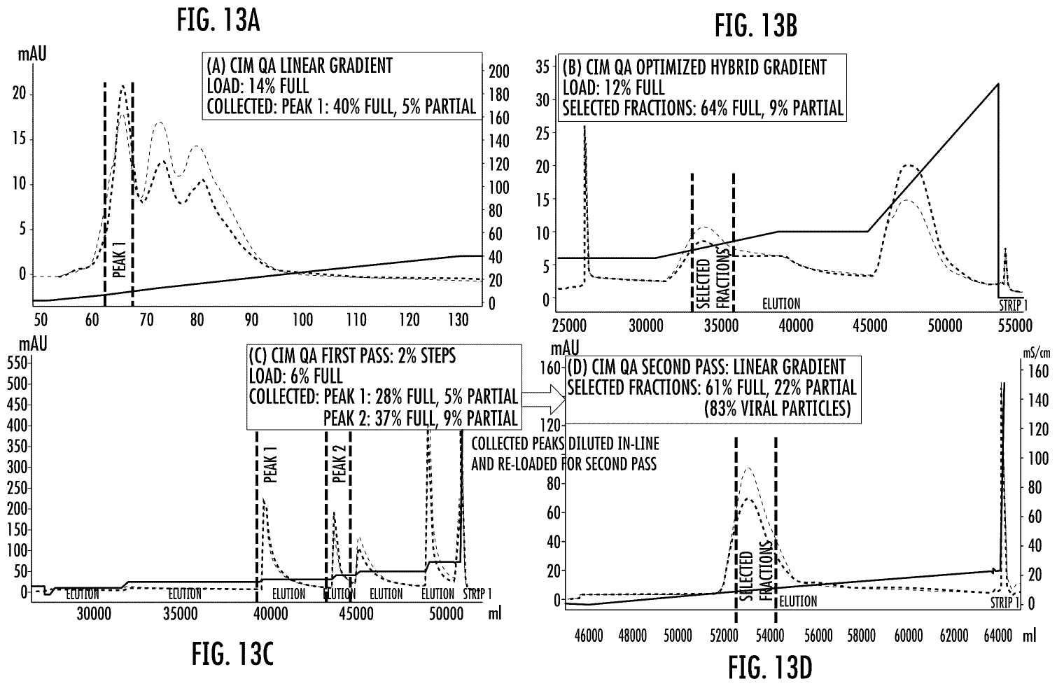

In this example, one-pass and two-pass AEX separation and purification of rAAV9 were undertaken and compared in terms of percent full capsids. See FIGS. 13A-13D. FIG. 13A shows results from a one AEX pass linear grade elution. FIG. 13B shows results from a one AEX pass elution using an optimized linear step gradient. FIG. 13C shows results from the first AEX pass of a two AEX pass method using a microstep approach. FIG. 13D shows the results of the second AEX pass of a two AEX pass method using a microstep approach. 1 ml CIM QA monolith columns were used. 5×10¹³ capsids/ml were use on all runs. “Full” capsids are considered functional in terms of a complete GOI and flanking ITRs, whereas “partial” or “partially-filled” capsids would not.

The system is as follows:
Buffer system: 20 mM BTP, 0.001% P188, pH 9.6 in Buffer A, and 20 mM BTP, 0.001% P188, pH 9.6, 1 M NaCl in Buffer B
Linear gradient: 0-40% B in 80 CV.
Elution microstep size selection 2% Buffer B based on dividing the linear gradient full peak elution range into 3 parts
Elution Step
Two pass: 2% microstep to 0-40% B in 80 CV Linear Gradient.

FIG. 13A depicts the results where the initial AAV load of 14% full capsids. A linear gradient was used. The enrichment achieved 40% full capsids and 5% partially-filled capsids.

FIG. 13B depicts the results where the initial AAV load had 12% full capsids. An optimized linear step gradient was used. The enrichment of selected fractions achieved 64% full capsids and 9% partially-filled capsids.

FIG. 13C depicts the results where the initial AAV load had 6% full capsids. A microsteps elution approach was used. The first AEX pass resulted in two elution peaks that fit the UV260/UV280 collection criteria. Peak 1 contain 28% full capsids and 5% partially-filled capsids. Peak 2 contained 37% full capsids and 9% partially-filled capsids.

FIG. 13D depicts the second AEX pass. Peak 1 and 2 were collected, diluted and subjected to the second AEX pass on the same column using a microsteps approach. Selected fractions contained 61% full capsids and 22% partially-filled capsids, which amounted to multi-fold enrichment in terms of full capsids from FIG. 13C.

### Example 7—Comparison of AEX Approaches with Recombinant AAV2

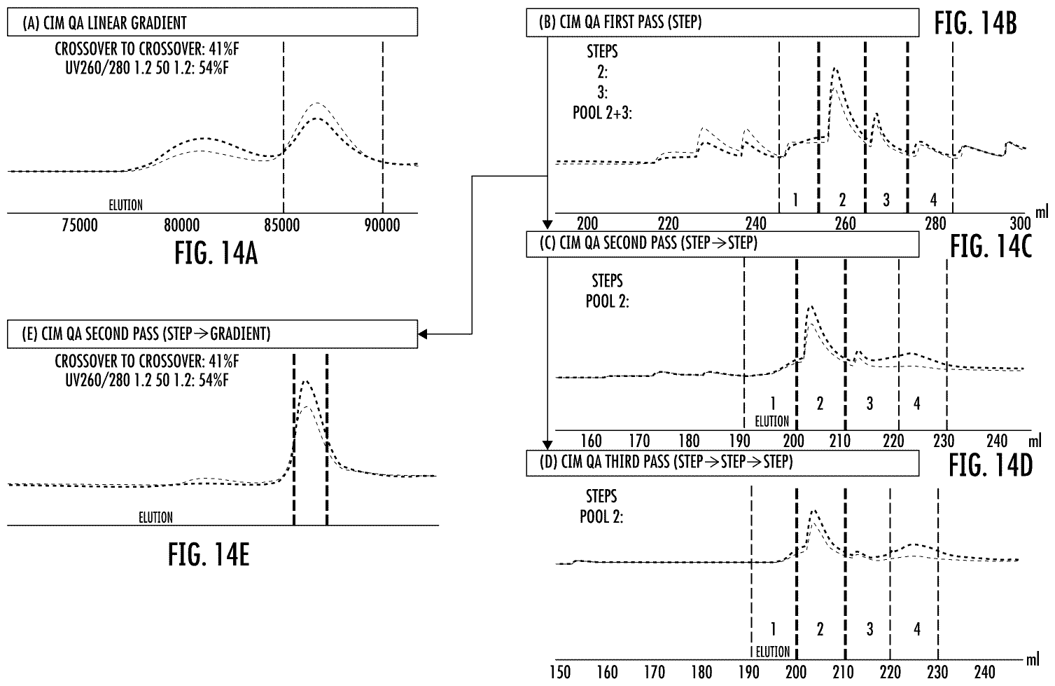

In this example, rAAV2 was subject to one, two or three AEX passes using a CIM QA AEX column. FIG. 14A is a graph depicting elution on a CIM QA Linear Gradient. FIG. 14B is a graph depicting a CIM QA First AEX Pass. FIG. 14C is a CIM QA Second AEX Pass using the pool from FIG. 14B. FIG. 14D is a CIM QA Third AEX Pass using the pool from FIG. 14C. FIG. 14E is a Second AEX Linear Gradient Pass using the pool of FIG. 14B.

The system is as follows:
Buffer system: 20 mM BTP, 1 mM MgCl₂, 0.001% P188, pH 9.0 in Buffer A, and 20 mM BTP, 20 mM MgCl₂, 0.001% P188, pH 9.0, 250 mM NaCl in Buffer B
Linear gradient: 0-50% B in 100 CV
Microstep size selection: 3.1% Buffer B based on dividing the linear gradient full peak elution range into 3 parts
Two pass 3.1% Elution Microstep to 3.1% Elution Microstep
Three pass 3.1% Elution Step to 3.1% Elution Microstep to 3.1% Elution Microstep.
Two pass 3.1% Elution Microstep to 0-50% B in 100 CV Linear Gradient.

### Example 8—System for Recombinant AAV8

A system for rAAV8 would be as follows:
Buffer system: 20 mM BTP, 0.001% P188, pH 8.5 for Buffer A, and 20 mM BTP, 0.001% P188, pH 8.5, 250 mM NaCl in Buffer B.
Linear gradient: 15-65% B in 120 CV.
Elution Step size selection: 2.4% Buffer B.
Two pass 2.4% Elution Microstep to 2.4% Elution Microstep.
Three pass 2.4% Elution Microstep to 2.4% Elution Microstep to 2.4% Elution Microstep.
Two pass 2.4% Elution Microstep to 15-65% B in 120 CV Linear Gradient.

### Example 9-Two-Pass Purification Comparisons using Single Modality and Orthogonal Modalities with Recombinant AAV9

Experimental characterization of two-pass purification was carried out for CIM QA columns (an anion exchange monolith) and Prima T columns (anion exchange and metal-ion affinity monoliths) in four combinations (CIM QA→CIM QA; CIM QA→Prima T; Prima T→CIM QA and Prima T→Prima T) with rAAV9. It was observed that mixing modalities led to a reduction in the yield of the second pass from about 80% to about 60%. This data below in Table 4 suggest that is best to re-load material on a single selected modality in the second pass rather than switch between modalities.

TABLE 4

| | % Viral Particles | | % Yield over | % Yield over | |
|---|---|---|---|---|---|
| | (% Full + % Partial | | First Pass | Second Pass | % Yield overall |
| | by Mass | | (by Droplet | (by Droplet | (by Droplet |
| AEX Strategy | Photometry) | | digital PCR) | digital PCR) | digital PCR) |
| CIM QA First Pass | 66% | (58% F, 8% P) | 42.5-47.8 | NA | 42.5-47.8 |
| Prima T First Pass | 52% | (46% F, 6% P) | 39.6 | NA | 39.6 |
| CIM QA First Pass→ PRIMA T | 86% | (73% F, 13% P) | 42.5-47.8 | 64.9 | 27.6-31.1 |
| Second Pass | | | | | |
| CIM QA First Pass→ CIM QA | 89% | (77% F, 12% P) | 42.5-47.8 | 85.6 | 36.4-40.9 |
| Second Pass | | | | | |
| PRIMA T First Pass→ CIM QA | 80% | (79% F, 1% P) | 39.6 | 65.1 | 25.8 |
| Second Pass | | | | | |
| PRIMA T First Pass → PRIMA T | 81* | (76% F, 5% P) | 39.6 | 80.9 | 32.0 |
| Second Pass | | | | | |

Load Details: 1.03×10¹² vg/ml, 12% full by MP (6 ml load meaning 6.12×10¹³ capsids loaded per 1 ml C/M QA/Prima T column).

CIM QA Method: 20 mM Bis-Tris-Propane (BTP), 0.001% P188, pH 9.6 and 1 M NaCl with microsteps elution

PRIMA T Method: 20 mM BTP, 0.001% P188, 1% Sucrose pH 8.5 and 50 mM MgCl2 with microsteps elution.

The system was as follows:
CIM QA Buffer system: 20 mM BTP, 0.001% P188, pH 9.6 in Buffer A, and CIM QA Buffer system: 20 mM BTP, 0.001% P188, pH 9.6, 1 M NaCl in Buffer B Prima T Buffer system: 20 mM BTP, 1% Sucrose w/v, 0.001% P188, pH 9.0 in

Buffer A, and 20 mM BTP, 1% Sucrose w/v, 0.001% P188, pH 9, 50 mM MgCl2 in Buffer B
CIM QA 2% Microsteps→CIM QA 2% Microsteps
CIM QA 2% Microsteps→Prima T 6% Microsteps
Prima T 6% Microsteps→CIM QA 2% Microsteps
Prima T 6% Microsteps→Prima T 6% Microsteps.

All combinations lead to similar enrichment of full capsids, but two passes using a single modality of columns result in higher yield than switching between modalities.

### Example 10—Two Pass Purification with Recombinant AAV9-SpyTag

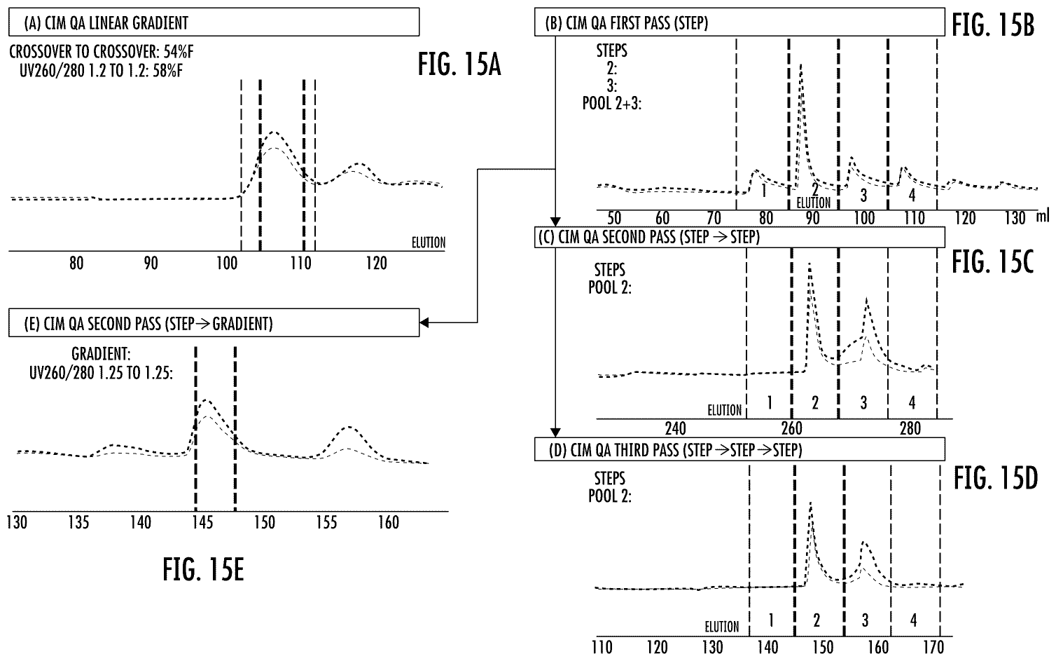

In this example, rAAV9-Spy Tag was subject to one, two or three AEX passes using a CIM QA AEX column. FIG. 15A is a graph depicting elution on a CIM QA Linear Gradient. FIG. 15B is a graph depicting a CIM QA First AEX Pass. FIG. 15C is a CIM QA Second AEX Pass using the pool from FIG. 15B. FIG. 15D is a CIM QA Third AEX Pass using the pool from FIG. 15C. FIG. 15E is a Second AEX Linear Gradient Pass using the pool of FIG. 15B.

The system is as follows:
Buffer system: 20 mM BTP, 0.001% P188, pH 9.6 in Buffer A, and 20 mM BTP, 0.001% P188, pH 9.6, 1 M NaCl in Buffer B
Linear gradient: 0-40% B in 80 CV.
Microstep size selection 2% Buffer B based on dividing the linear gradient elution range into 3 parts
Two pass 2% Microstep to 2% Microstep
Three pass 2% Microstep to 2% Microstep to 2% Microstep
Two pass 2% Microstep to 0-40% B in 80 CV Linear Gradient

### Example 11—One, Two and Three Pass Purification with Recombinant AAV9-SpyTag-SpyCatcher-FEL D1 Monoclonal Antibody on Prima T with Dual Mg Gradients

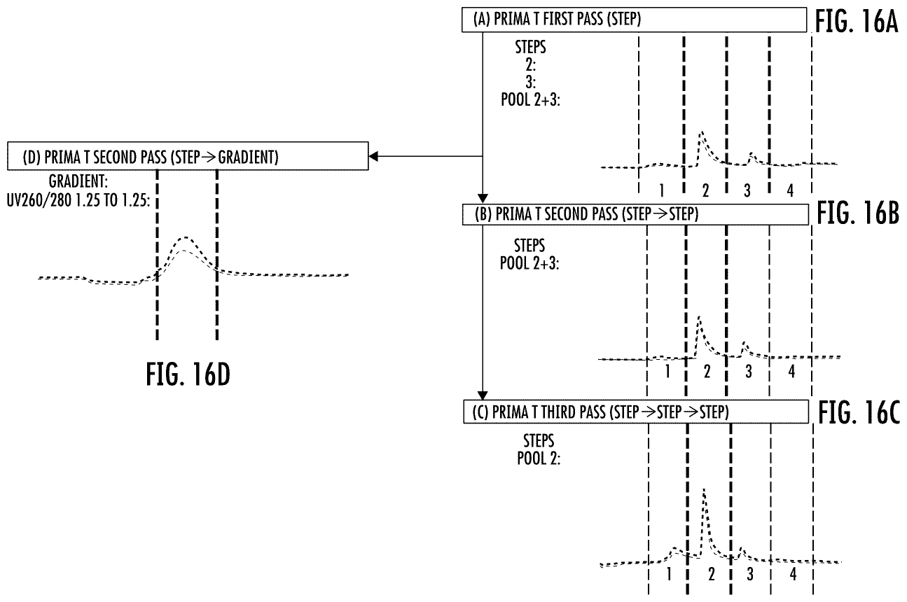

In this example, rAAV9-Spy Tag-Spy Catcher-FEL D 1 Ab was subject to one, two or three AEX passes using a Prima T AEX and metal ion affinity column. FIG. 16A is a graph depicting a Prima T First AEX Pass. FIG. 16B is a Prima T Second AEX Pass using the pool from FIG. 16A. FIG. 16C is a Prima T Third AEX Pass using the pool from FIG. 16B. FIG. 16D is a Second AEX Linear Gradient Pass using the pool of FIG. 16A.

The system is as follows:
20 mM BTP, 1% Sucrose w/v, 0.001% P188, pH 9.0 in Buffer A, and 20 mM BTP, 1% Sucrose w/v, 0.001% P188, pH 9.0, 100 mM MgCl₂, 40 mM MgSO₄ in Buffer B on Prima T
Linear gradient: 0-100% B in 50 CV.
Microstep size selection: 4% Buffer B based on dividing the linear gradient elution range into 3 parts
Two pass 4% Elution Microstep to 4% Elution Microstep
Three pass 4% Microstep to 4% Microstep to 4% Microstep.
Two pass 4% Microstep to 0-100% B in 50 CV Linear Gradient

### Example 12—Two Pass Purification with Recombinant AAV9-SpyTag-SpyCatcher-Tfr Fab

The system would be as follows:
Buffer system: 20 mM BTP, 1% Sucrose w/v, 0.001% P188, pH 9.0 in Buffer A, and 20 mM BTP, 1% Sucrose w/v, 0.001% P188, pH 9.0, 100 mM MgCl2, 40 mM MgSO₄ in Buffer B on Prima T
Linear gradient: 0-100% B in 50 CV.
Microstep size selection 4% Buffer B as per the teaching contained herein
Two pass 4% Microstep to 4% Microstep
Three pass 4% Microstep to 4% Microstep to 4% Microstep.
Two pass 4% Microstep to 0-100% B in 50 CV Linear Gradient

### Example 13—Two Pass Purification with rAAV2

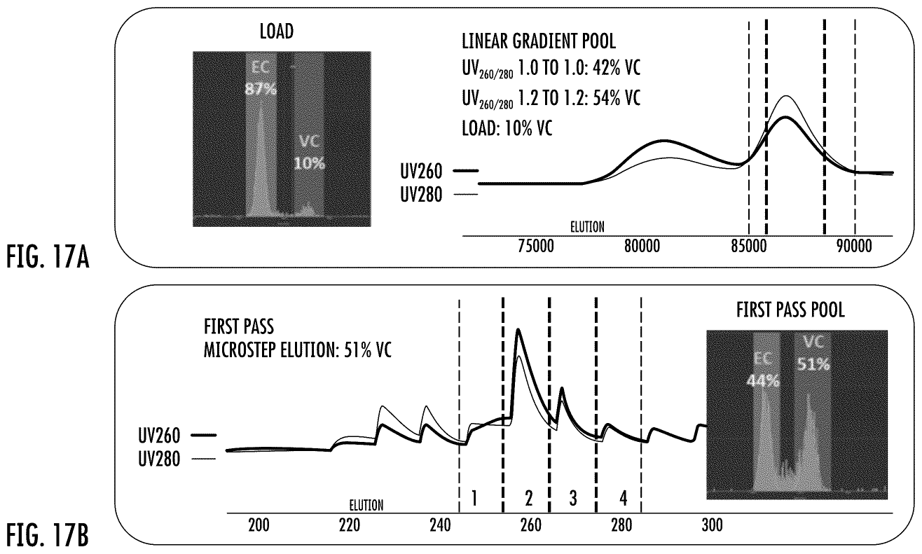

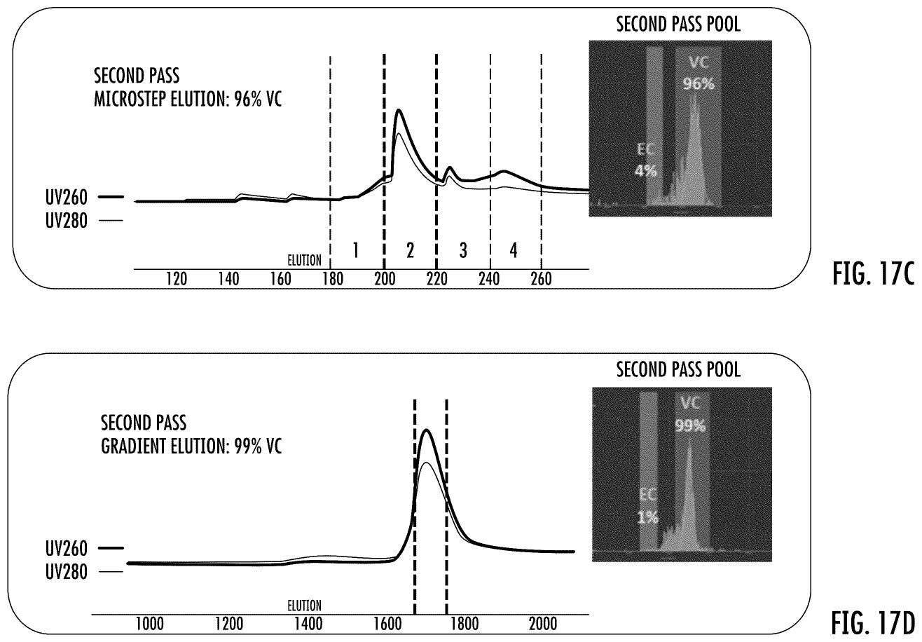

FIGS. 17A-17D provide another comparison of percent full achieved by one AEX pass and two AEX pass methods using rAAV2. FIG. 17A is a graph depicting elution on a CIM QA Linear Gradient. FIG. 17B is a graph depicting a CIM QA First AEX Pass. FIG. 17C is a CIM QA Second AEX Pass using microsteps using the pool from FIG. 17B. FIG. 17D is a CIM QA Second AEX Pass using linear gradient the pool from FIG. 17B. Mass photometry histograms depicting populations of full and empty capsids are displayed for the load, first pass pool, and second pass pools.

As used throughout in the description of the inventions, “EC” stands for an “Empty Capsid,” also referred to as “Empty.” “VC” stands for “Full Viral Capsid,” also referred to as “Full.”

### Example 14—Additional Two-Pass Purification Analyses

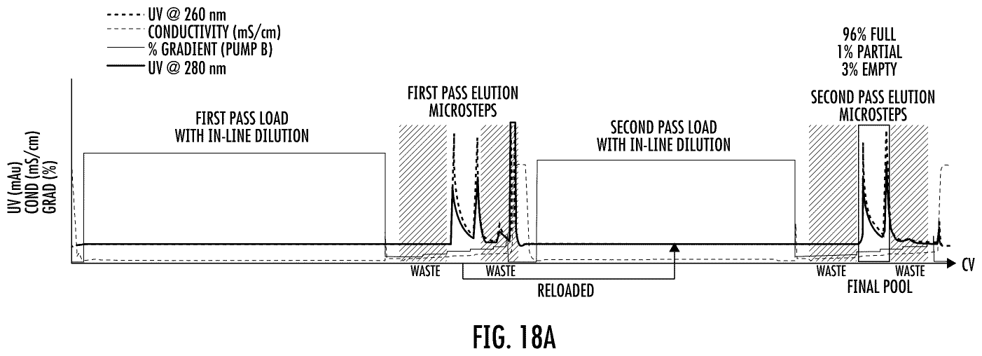

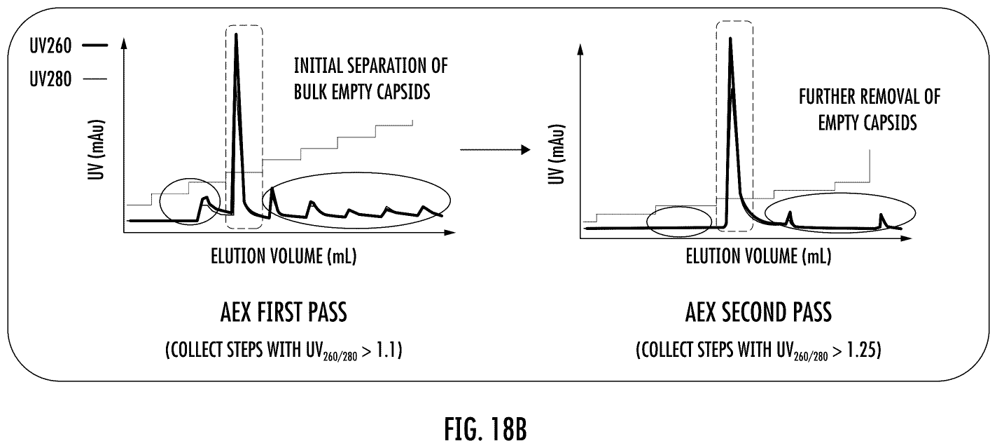

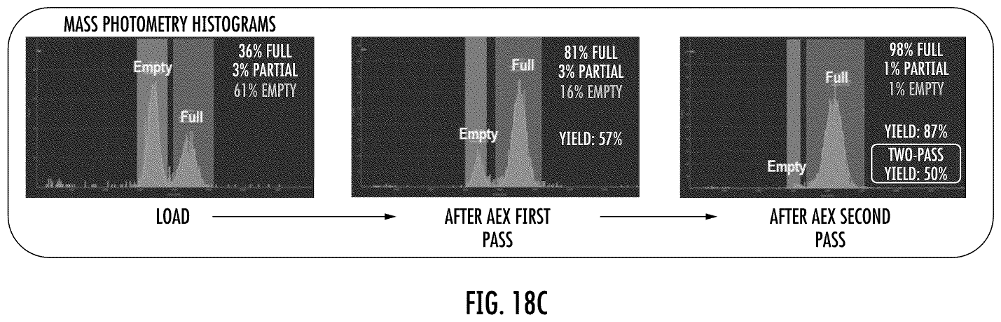

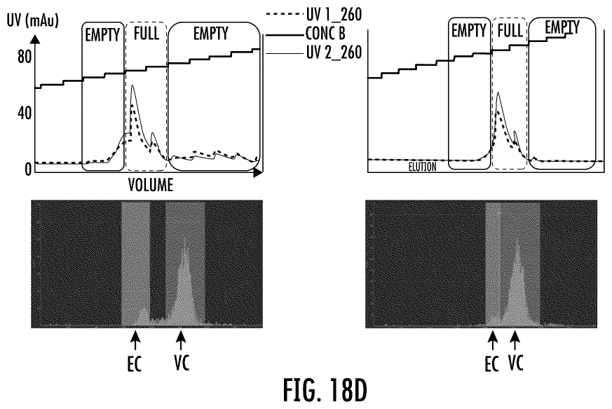

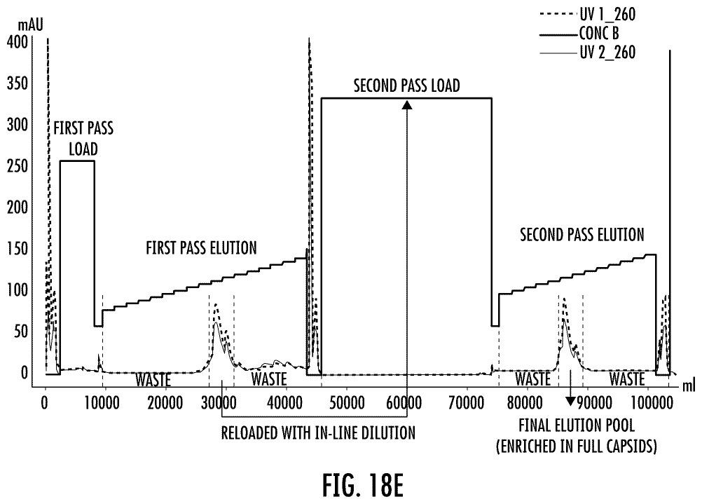

FIG. 18A depicts data from a two-pass anion exchange chromatogram for a 500 L purification of AAV-SpyTag using first pass and second pass microsteps. This process achieved a recovery of 96% full capsids. Partially-filled capsids were only 1% of total and empty capsids were only 3% of total. FIG. 18B depicts initial separation of bulk empty capsids in a first AEX pass followed by further removal of empty capsids in an AEX second pass. FIG. 18C depicts mass photometry data showing an enrichment from 36% full capsids to 81% full capsids after a first AEX pass to 98% full capsids after a second AEX pass.

FIG. 18D depicts small scale purification of rAAV1 (1 ml scale) with histograms on top and mass photometry chromatograms below, where the first pass of AEX resulted in an enriched pool of full rAAV1 capsids and the second pass of AEX resulted in a further enriched pool of full rAAV1 capsids. FIG. 18E depicts a pilot scale chromatogram of rAAV1 in a 50 liter bioreactor using an automated two-pass Akta AEX Pilot 600 system using 400 ml of a CIM QA HR monolith. First and second pass data at the 1 ml and 400 ml scales is set forth in Table 5.

TABLE 5

| | Scale (CIM | % Viral Capsids | % Genomic Yield |
|---|---|---|---|
| | QA HR Column | by Mass | over AEX Pass |
| Entity | Volume, mL) | Photometry | by ddPCR |
| Load | — | 37 | — |
| AEX First Pass | 1 mL | 87 | 55% |
| Pool | | | |
| AEX Second | 1 mL | 97 | 84% |
| Pass Pool | | | |
| AEX First Pass | 400 mL | 91 | 60% |
| Pool | | | |
| AEX Second | 400 mL | 96 | 87% |
| Pass Pool | | | |

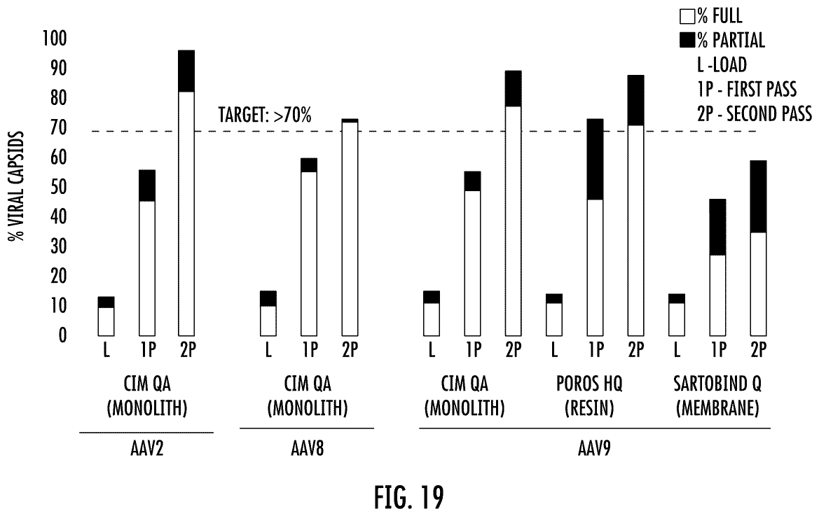

FIG. 19 depicts a comparison of percent full achieved by one AEX pass and two AEX pass methods using AAV2, AAV8 and AAV9-SpyT on CIM QA monolith, POROS 50 HQ resin, and Sartobind Q membrane chromatographic modalities. The target of greater than 70% full capsids is achieved for all three serotypes using CIM QA monolith, and also on Poros 50 HQ resin for AAV9-SpyT, but not using Sartobind Q membranes for AAV9-SpyT.

### Example 15—Process Analytical Technology and Automation

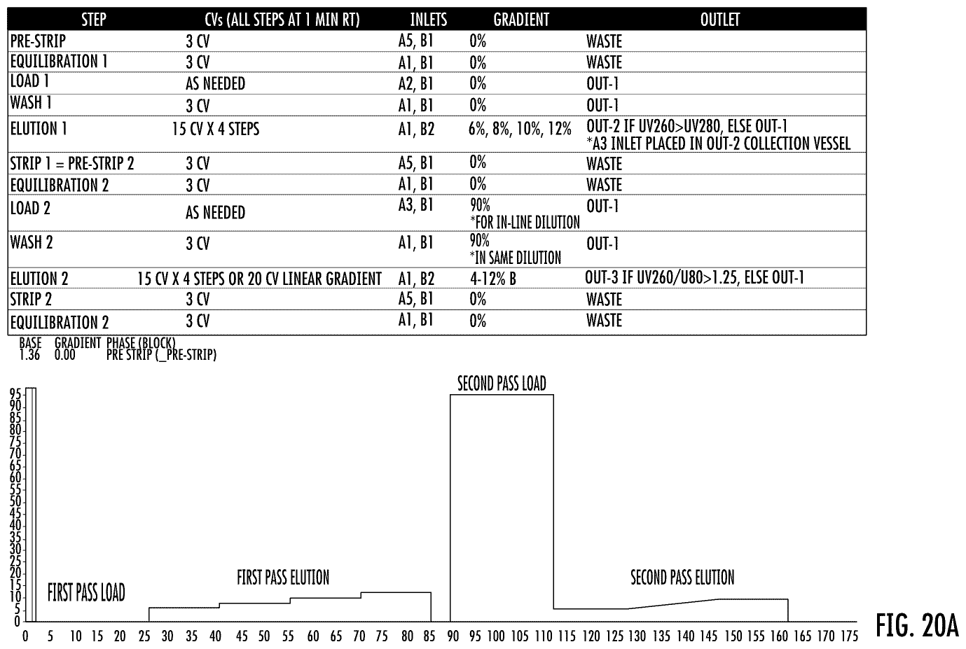

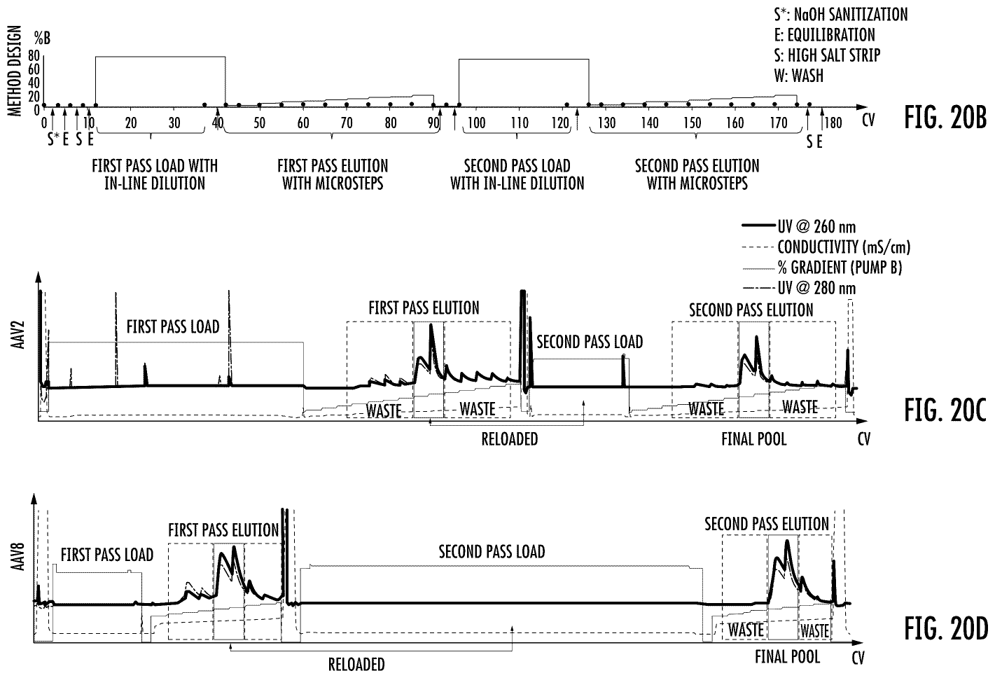

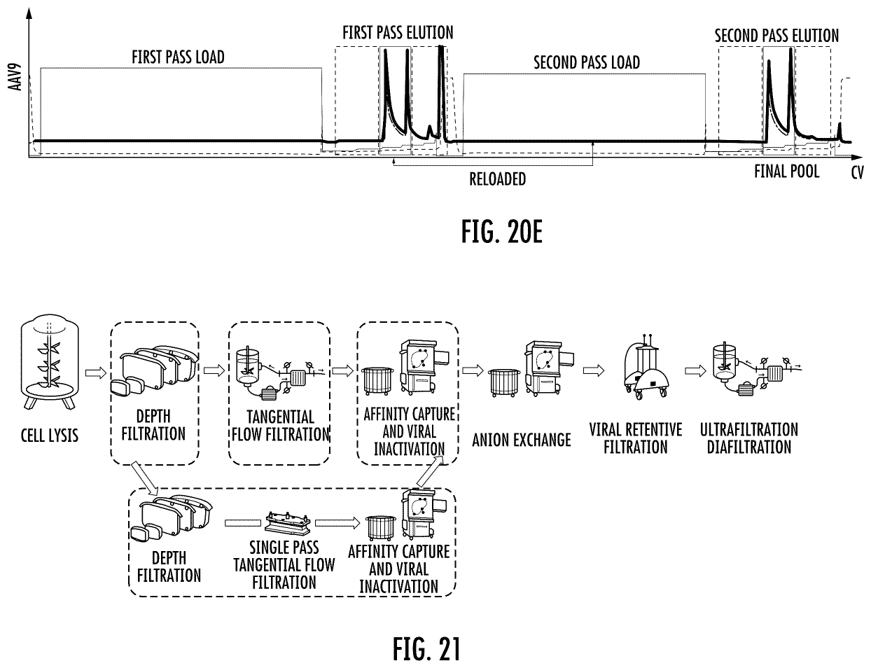

An automated approach for a two AEX pass method using Atka Avant, Pilot and Ready Extended chromatography systems, manufactured by Cytiva, is depicted in FIG. 20A. The automated approach is capable of in-line dilution for the second AEX pass without the need for manual intervention between enrichment steps. The top of FIG. 20A provides a table setting forth methodologies and parameters.

The graph at the bottom of FIG. 20A provides loading and elution information. The left side of the graph shows the data from the first load and first pass elution (collectively, “the first AEX pass”) and the right side of the graph shows second pass load and the second pass elution (collectively, “the second AEX pass”).

FIG. 20B illustrates a method design for an automated two-pass AEX method using microsteps for both the first and second passes on an Atka Pilot 600 system. FIG. 20C depicts a two-pass AEX chromatogram for AAV2 carried out at 50 L bioreactor scale on a 400 mL CIM QA monolith, resulting in a final pool with >70% full capsids. FIG. 20D depicts a two-pass AEX chromatogram for AAV8 carried out at 50 L bioreactor scale on a 80 mL CIM QA monolith, resulting in a final pool with >70% full capsids. FIG. 20E depicts a two-pass AEX chromatogram for AAV9-SpyT at 500 L bioreactor scale on a 400 mL CIM QA monolith, resulting in a final pool with 96% full and less than 1% partial capsids as measured by mass photometry.

### Example 16—Purification Trains and Components

FIG. 21 depicts exemplary production purification trains for AAV, such as recombinant AAV. The top train uses a batch process where repeated passes are required to exchange buffer and concentrate the retentate, which contains the desired biological material, such as AAV. See Adams et al., Biotech. Bioeng. 117:3199-3211 (2020).

The bottom section of FIG. 21 replaces the batch tangential flow filtration unit with a single-pass tangential flow filtration unit (SPTFF unit), which permits a continuous process. It was surprising how well SPTFF performed with AAV, as taught herein,

The Batch TFF approach can take multiple days (for example, 2 days) due to the repeated cycling through the conventional TFF unit to achieve concentration prior to further purification. The SPTFF approach is a continuous approach, and is significantly faster than the Batch TFF approach, and can be performed in several hours, such as 3 to 5 hours. The SPTFF approach provides faster concentration, while minimizing sheer stress and damage to AAVs.

An exemplary approach can include:
Day 1—Lysis, clarification, SPTFF and Affinity Capture;
Day 2—Anion exchange;
Day 3—Genomic titer analysis;
Day 4—Formulation and in vitro conjugation (in the case of using specific binding pairs, such as SpyTag-SpyCatcher); and
Day 5—Purification to remove free antibodies, such as by chromatography (for example, ionic exchange) followed by final formulation.

The SPTFF approach also is amenable to the use of Process Analytical Technology (PAT) and automation.

For further comparison, FIG. 22A schematically shows a Batch TFF (top), where the retentate is repeatedly cycled through a feed tank and pump to repeatedly passed through a membrane, with the concentrated retentate being removed after repeated cycles. A Single-Pass TFF (bottom) removes biological material from the feed tank through a pump to a multi-stage membrane module that separate the retentate from the permeate, while concentrating the retentate.

FIG. 22B is a graph comparing Batch TFF and Single-Pass TFF. Single-Pass TFF achieves higher concentration and is faster as compared to Batch TFF. Single-Pass TFF continuously sends biological material to the next operation in the purification train, whereas Batch TFF does not send biological material until the end of the batch cycle.

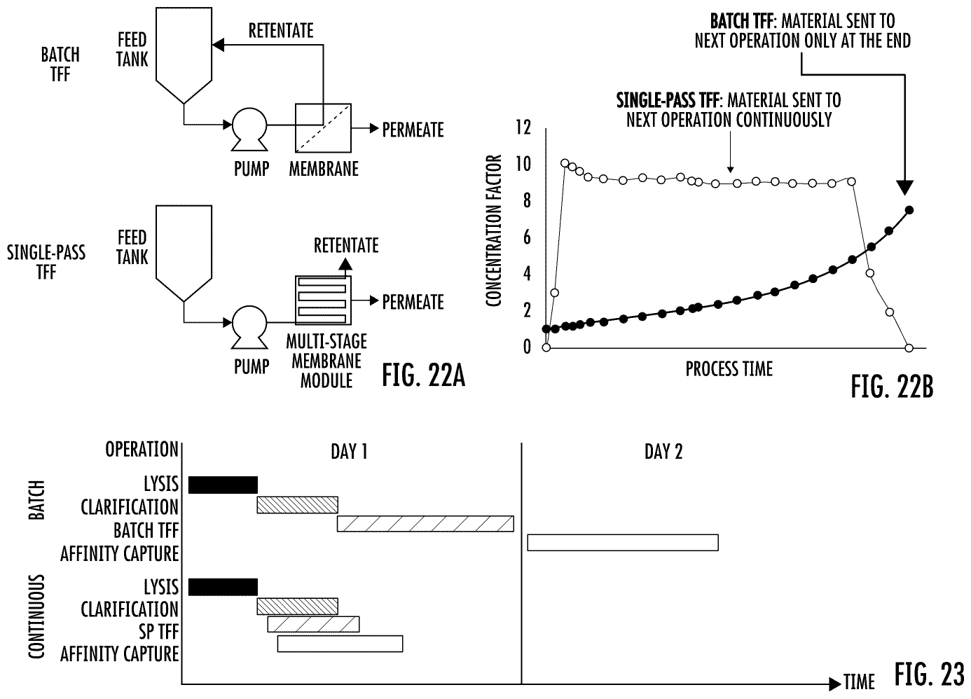

FIG. 23 schematically and qualitatively compares the batch operation to a continuous operation for AAV purification in terms of Cell lysis, Clarification (depth filtration), TFF (Batch or Single-Pass) and Affinity Capture. The continuous process (SPTFF) can be completed in less than a day, whereas the batch process can be multi-day.

The cell lysis step, typically using a detergent such as Tween-20, typically takes up to about two hours, and is depicted as the same for both the Batch and the SPTFF (continuous) process. Following lysis, clarification takes about 1 hour. The processes then diverge at the TFF step.

For the Batch process, TFF takes about 3 hours per batch due to the repeated cycling. Not until a batch is complete can the concentrated biological material in a buffer be passed on the affinity capture, which takes about 2 to 3 hours per batch. Because multiple batches are required, the affinity chromatography is typically not completed until the next day.

For the Continuous process, clarification, SPTFF and affinity capture can take place substantially simultaneously. Biological material continuously flows to clarification (about 1 hour), SPTFF (about 1½ hours) and affinity capture (about 2 hours to 3 hour). Accordingly, when an early portion of biological material is in affinity capture, later portions of biological material are in SPTFF or clarification.

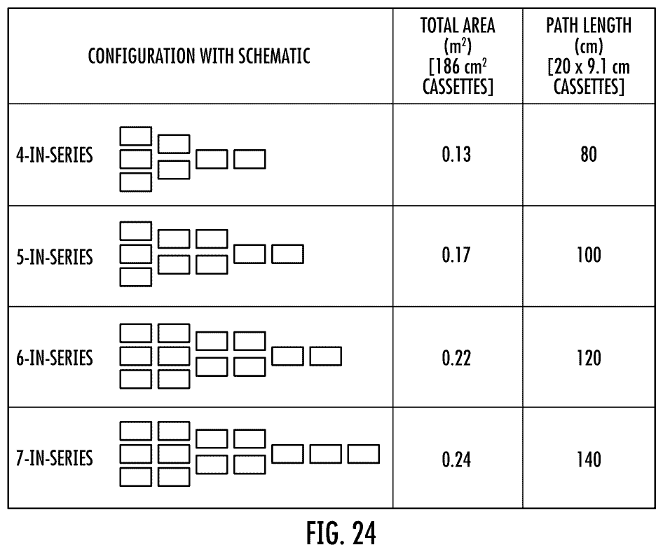

FIG. 24 schematically depicts exemplary arrangements for multi-stage membrane module cassettes to be used with Single-Pass TFF. The configurations depict four to seven tiers of membrane module cassettes where the initial tiers (left side) contain more or same number of membrane module cassettes as the succeeding tiers (moving towards the right side), in the manner suggested by the manufacturer, here Pall/Cytiva. Total area and path length of the membrane module cassettes also are set forth. Other arrangement of membranes, flow rates and transmembrane pressure (TMP) can be selected by the person skilled in the art.

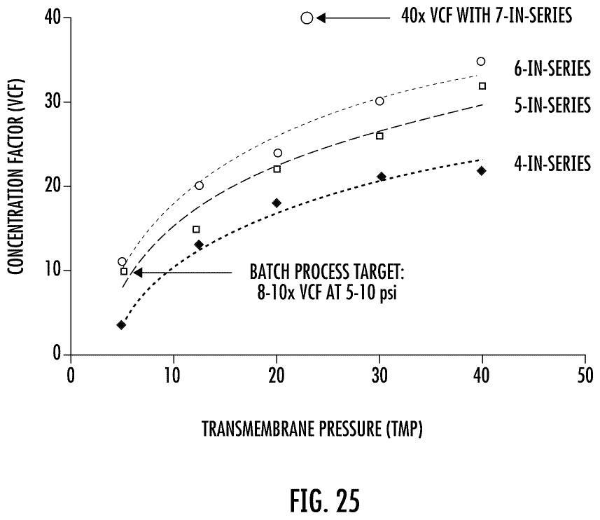

FIG. 25 is a graph depicting volumetric concentration factor (VCF) versus transmembrane pressure (TMP) using the 4-in-series, 5-in-series, 6-in-series and 7-in-series exemplary configurations depicted in FIG. 24 with a feed comprising an exemplary AAV, here AAV9 comprising a SpyTag insert. A Batch process target would be 8-10×VCF at a TMP of 5 to 10 psi.

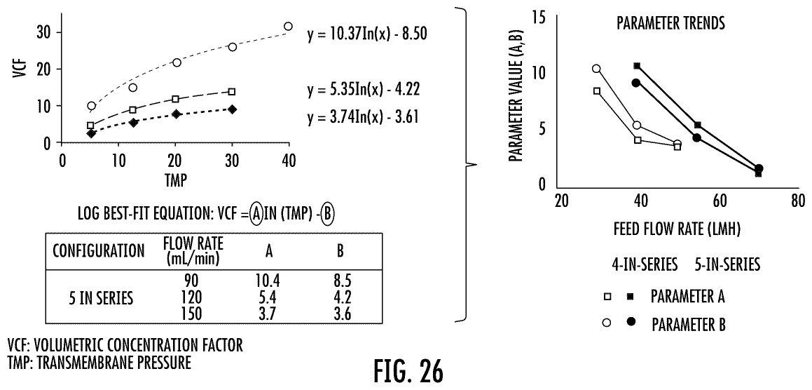

FIG. 26 depicts data from a 5-in-series configuration according to FIG. 24 at flow rates of 90 ml/minute, 120 ml/minute and 150 ml/minute. The log best-fit equation of VCF=A In (TMP-B) using the values at each flow rate set forth near the plot (and rounded off in the chart) can be used to parameterize the data. At the right side of the figure, there is a graph of parameter value (A, B) and feed flow rate in liters per square meter of membrane per hour (LMH) for 4-in-series and 5-in-series exemplary configurations of FIG. 24 and allows optimized conditions to be selected in silico using an exemplary AAV, here AAV9 comprising a SpyTag insert. This model can be used to predict the VCF for any flow rate and TMP for an in-series configuration of interest.

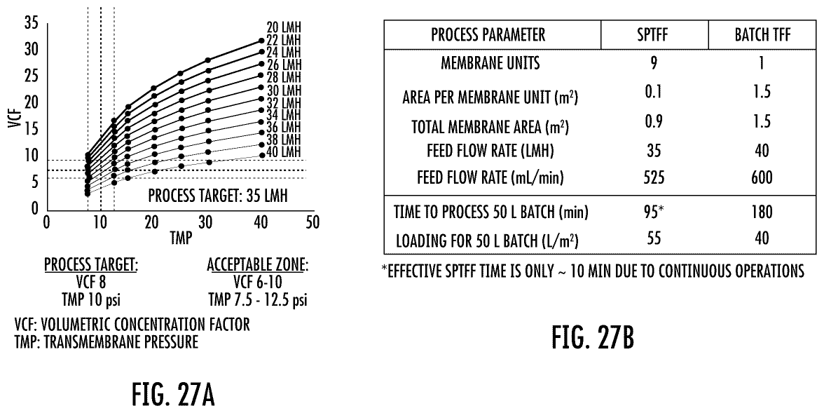

FIG. 27A is a design space model based on FIGS. 22A, 22B and 23 using the 5-in-series configuration of FIG. 24. Here, the process target was 35 LMH, and the intersecting lines indicate a VCF of 8 and a TMP of 10 psi. An exemplary acceptable zone would be a VCF of 6-10 and a TMP of 7.5 to 12.5 psi. FIG. 27B is an exemplary comparison of process parameters between SPTFF and Batch TFF. With Batch TFF, typically there would be one batch before the next operation. However, depending on the scheduling of upstream production bioreactors and bioreactor titers, there could be pooling of multiple batches before the next operation. Effective residence time of a given portion of biological material in the SPTFF is only about 10 minutes, and the overall time is for all biological material to pass thought the SPTFF.

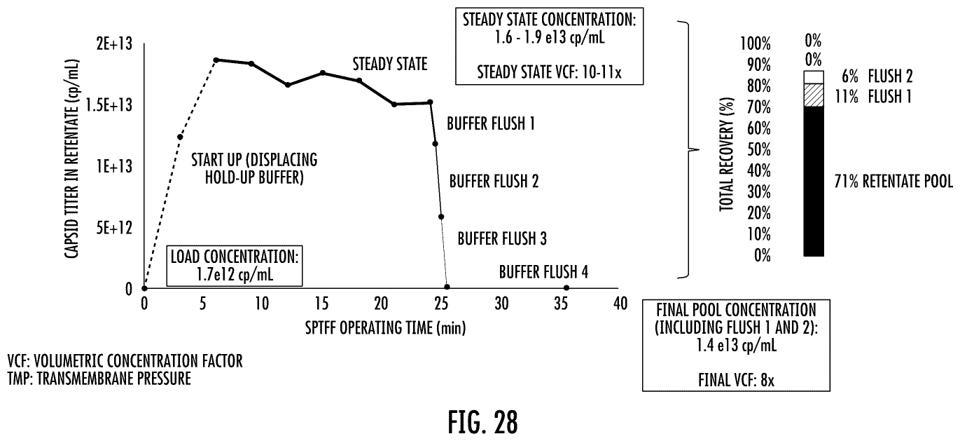

FIG. 28 depicts data from a bench-scale trial to determine the number of buffer washes need to attain about a 90% recovery of AAV, here AAV9 with integrated SpyTag, in a low-TMP process. On average, the AAV9 here contained 6 SpyTag peptides per viral capsid. The load concentration was 1.7×10¹² capsids/ml (cp/ml). The steady state concentration using SPTFF was 1.6−1.9×10¹³ cp/ml, yielding a steady state VCF of 10 to 11×. Capsid titer in retentate (cp/ml) versus SPTFF operating time (minutes) was measured using four buffer flushes. The final pool flush (1 and 2) achieved 1.4×10¹³ cp/ml. As the right side of the figure shows, 71% of capsids were recovered in the retentate pool, 11% of capsids were recovered by flush 1 and 6% of capsids were recovered with flush 2. It was determined that only two buffer flushes were required to achieved about a 90% recovery with a VCF of 8×.

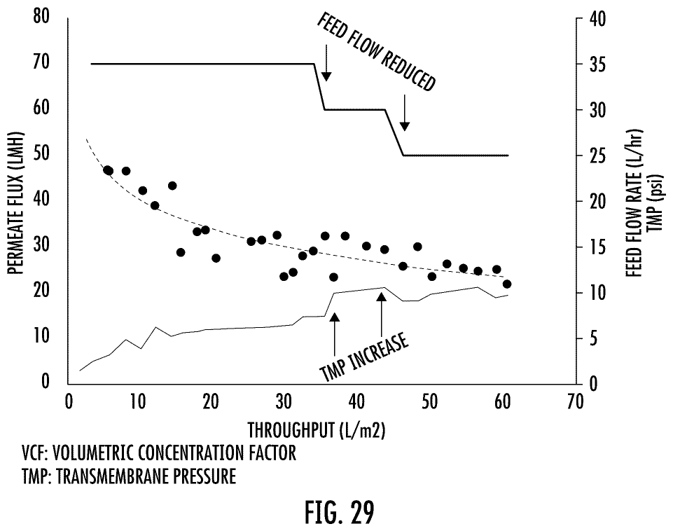

FIG. 29 is a graph depicting Permeate Flux (LMH), Throughput (L/m²), Feed Flow Rate (L/hr) and TMP (psi) in a pilot-scale trial. Using continuous SPTFF, the data showed flux decline and TMP build up. To mitigate TMP increase beyond 12.5 psi, feed flow rate was slowed. This resulted in a longer process time of 180 minutes rather than the expected 90 minutes and an overall VCF of 5×was achieved rather than the target VCF of 8×.

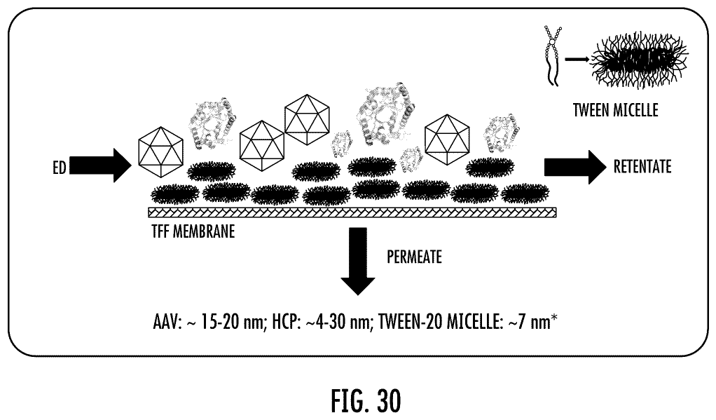

FIG. 30 depicts a tween micelle build-up on the TFF membrane. Without being bound by any theory or hypothesis, it is believed that detergent micelle buildup (here, Tween-20) is the cause of an unexpected flux decline of about 50% using SPTFF to concentrate AAV. Typically, a 20% flux decline is expected when concentrating antibodies. This figure also set forth the approximate size of AAV, Host Cell Protein aggregates (HCP) and Tween-20 micelles. The micelle concentration of Tween-20 was determined to be about 0.7%. See Basheva et al, J. Physical Chemistry Chemical Physics, Issue 38 (2007) (discusses properties of films formed by Brij 35 and Tween 20). Detergents, such as Tweens, are a common component of cell lysis buffers used in the production of AAV.

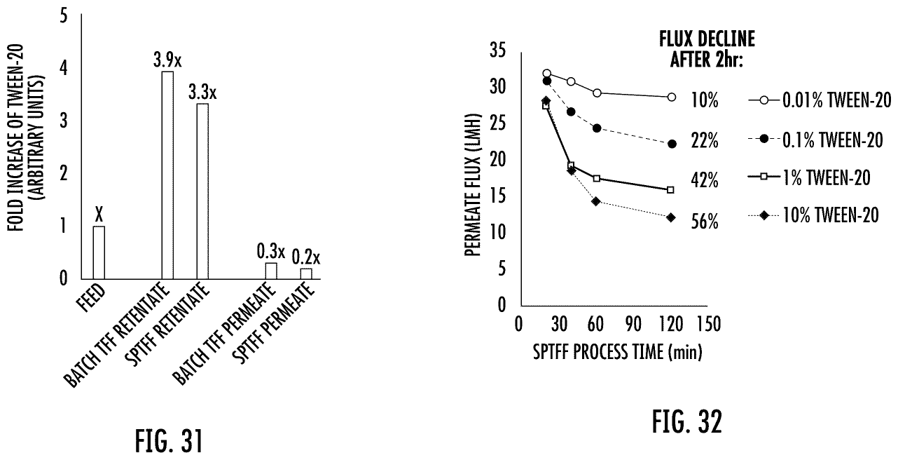

FIG. 31 is a graph depicting fold presence of Tween-20 on the retentate side of membrane and the permeate side of the membrane for both Batch TFF and SPTFF. Most Tween-20 is on the retentate side.

FIG. 32 is a graph depicting the flux decline after two hours with varying percentages of Tween-20 in the lysis buffer. The lower the percentage of Tween-20, the lower the percentage of flux decline encountered. In addition to Tween-20, the buffer contained 20 mM Tris, 2 mM MgCl₂ at a pH of 7.4. The feed flow rate was 35 LMH and the TMP was about 5 to 10 psi.

With Batch TFF, flux decline can be address by increasing processing time. However, with SPTFF immediate control is desired.

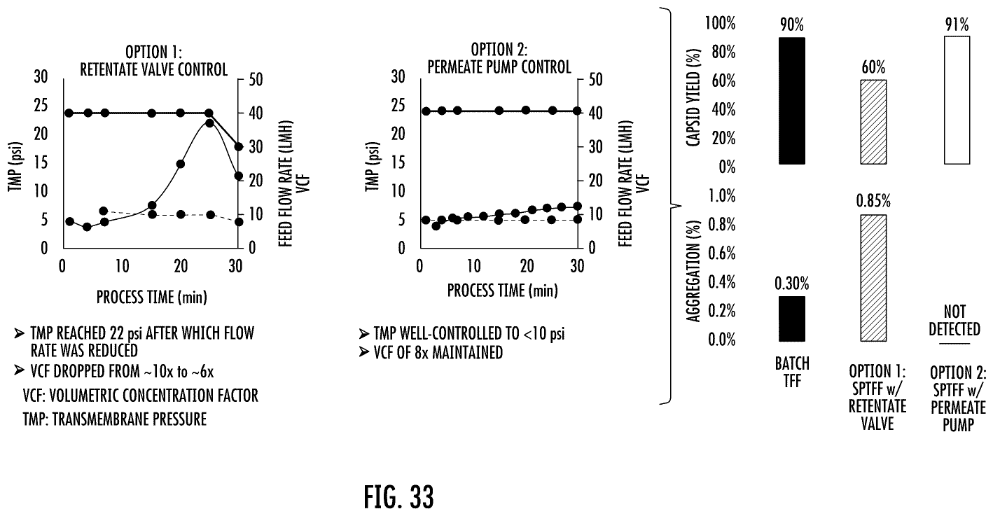

FIG. 33 compares control with the retentate valve to control with a Permeate pump. Option 1 with the retentate valve found that TMP reached 22 psi, and after which the flow had to be reduced from 40 LMH to 30 LMH. VCF dropped from about 10× to about 6×. Option 2 with the permeate pump was superior, which acts as a suction pump. TMP was controlled to well under 10 psi and a VCF of 8× was maintained. At the right side to the figure Option 1 (SPTFF with retentate valve) and Option 2 (SPTFF with permeate pump) were compared to a Batch TFF. Option 1 did not perform as well as Option 2 and Batch TFF. Option 2 was superior to Batch TFF and Option 1 in terms of capsid yield and percent aggregation. The permeate pump flow should be set within the VCF design space to avoid negative permeate pressure buildup. Thus, the flow rate of operation of the permeate pump should be within the range established in FIG. 27A.

FIG. 34 depicts the overall pilot scale process, and has similarities to parts of the production process of FIG. 21.

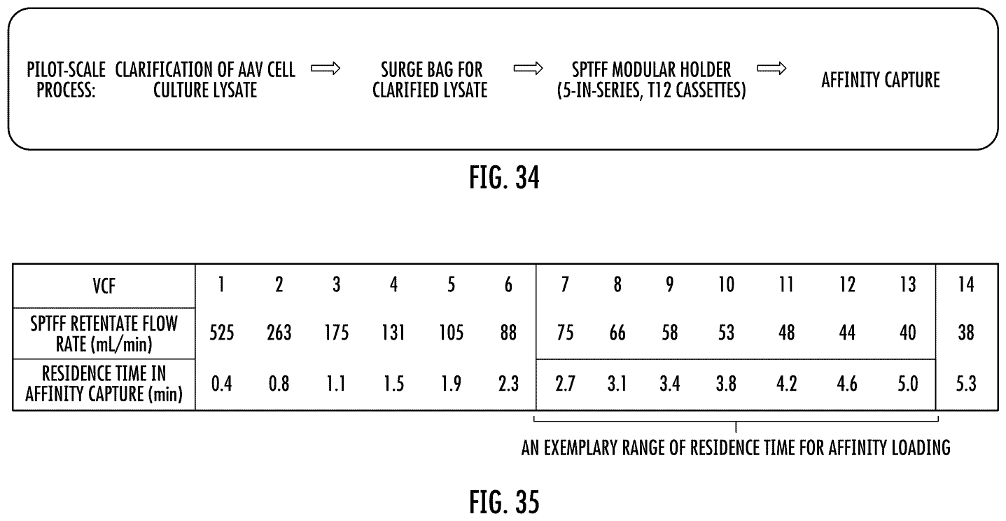

FIG. 35 compares VCFs (1-14), SPTFF retentate flow rates and residence time in affinity capture. VCFs of 7 to 13 and SPTFF retentate flow rates of 75-40 provided an exemplary range of residence time suitable for affinity loading. The flow rate should be selected to avoid depleting or overwhelming the affinity column. This calculation was based on a pilot-scale trial with a 525 ml/minute feed flow in a 5-in-one series SPTFF module and then loaded on to a 200 ml POROS CaptureSelect AAV9 column.

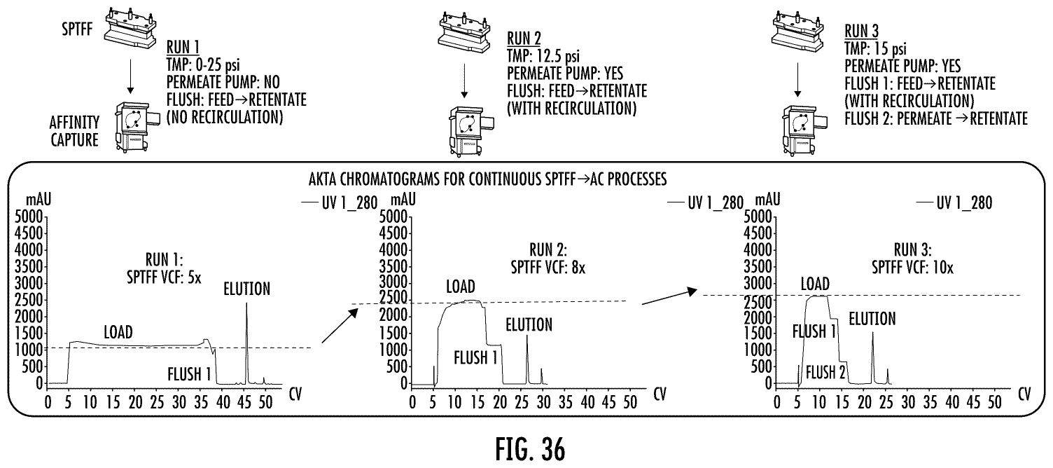

FIG. 36 depicts how UV280 profile of affinity capture flow can be used for process monitoring of VCF and process stability using SPTFF for continuous processing. Three different runs were performed for comparison purposes. Run 1 was performed without a permeate pump and achieved a VCF of only 5×. Run 2 was performed with a permeate pump with a feed to retentate flush (with recirculation) and achieved a VCF of 8×. Run 3 was performed with a permeate pump with a feed to retentate flush (with recirculation) and a permeate to retentate flush, which achieved a VCF of 10×. Most chromatography systems have built-in UV280 sensors that can detect load concentration, and provide an indication of VCF and process stability. Any needed correction, such as pump and/or valve control, can be based upon the data received through process analytical technology. See Thakur et al., J. Membrane. Sci. 613:118492 (2020) discuss the use of process analytical technology with SPTFF.

Advantages and Aspects of SPTFF include:
Greater efficiency in AAV manufacturing from harvest to final capture and purification;
Use of a permeate pump provides real-time control over TMP and maximizes AAV yield and minimizes AAV aggregation;
Detergents, such as Tween-20, can decrease permeate flux, which can be best managed through use of a permeate pump; and
Volumetric concentration factor (VCF) depends on flow rate, TMP and SPTFF membrane module configuration, which can be addressed by the empirical modeling of VCF vs. TMP curves for optimization based upon the teachings contained herein.

It is to be understood that the description, specific examples and data are given by way of illustration and are not intended to limit the present inventions. Various changes and modifications within the present inventions, including combining features in whole or in part, will become apparent to the skilled artisan from the discussion, disclosure and data contained herein, and thus are considered part of the inventions.

1. A method of purifying full adeno-associated virus (AAV) capsids, wherein the method comprises the steps of
(a) subjecting a sample comprising AAV capsids to anion exchange chromatography in a buffer using a microstep or a linear gradient to form a first pool, wherein the first pool is enriched in the ratio of full capsids to capsids that are not full; and
(b) subjecting the first pool to anion exchange chromatography from step (a) in a buffer using a microstep or a linear gradient to form a second pool, wherein the second pool is further enriched in the ratio of full capsids to capsids that are not full.

2. A method of purifying full adeno-associated virus (AAV) capsids, wherein the method comprises the steps of
(a) subjecting a sample comprising AAV capsids to depth filtration to form a first pool;
(b) subjecting a first pool from step (a) to a purification procedure and then anion exchange chromatography in a buffer using a microstep or a linear gradient to form a second pool, wherein the second pool is enriched in the ratio of full capsids to capsids that are not full; and
(c) subjecting the second pool from step (b) to anion exchange chromatography in a buffer using a microstep or a linear gradient to form a third pool, wherein the third pool is further enriched in the ratio of full capsids to capsids that are not full.

3. The method according to claim 2, wherein the depth filtration does not require an endonuclease.

4. A method of purifying full adeno-associated virus (AAV) capsids,
wherein the method comprises the steps of (a) subjecting a sample comprising AAV capsids to depth filtration to form a first pool;
(b) subjecting the first pool from step (a) to affinity chromatography to form a second pool;
(c) subjecting the second pool from step (b) to anion exchange chromatography in a buffer using a microstep or a linear gradient to form a third pool, wherein the third pool is enriched in the ratio of full capsids to capsids that are not full; and
(d) subjecting the third pool from step (c) to anion exchange chromatography in a buffer using a microstep or a linear gradient to form a fourth pool, wherein the fourth pool is further enriched in the ratio of full capsids to capsids that are not full.

5. The method according to claim 4, wherein the depth filtration does not require an endonuclease.

6-15. (canceled)

16. The method according to claim 1, wherein the same anion exchange chromatography unit can be used for step (a) and step (b).

17-18. (canceled)

19. The method according to claim 1, where steps (a) and (b) are executed on the same chromatography unit.

20. The method according to claim 4, wherein steps (a), (b) and (c) are executed on the same chromatography unit.

21. The method according to claim 1, where steps (a) and (b) are executed on different chromatography units.

22. The method according to claim 4, wherein steps (a), (b) and (c) are executed on different chromatography units.

23-31. (canceled)

32. The method according to claim 1, wherein the AAV virus is a recombinant AAV virus comprising a gene of interest flanked by AAV inverted terminal repeats.

33. The method according to claim 32, wherein the gene of interest encodes a protein of interest selected from the group consisting of viral proteins, bacterial proteins, fungal proteins, plant proteins and animal proteins.

34. The method according to claim 33, wherein the gene of interest encodes a human protein.

35. The method according to claim 34, wherein the gene of interest encodes a protein of interest selected from the group consisting of antibodies, receptors, Fc-containing proteins, trap proteins, mini-trap proteins, fusion proteins, antagonists, inhibitors, enzymes, factors, repressors, activators, ligands, reporter proteins, selection proteins, protein hormones, protein toxins, structural proteins, storage proteins, transport proteins, neurotransmitters and contractile proteins.

36-37. (canceled)

38. The method according to claim 1, wherein the AAV virus is a covalently surface modified AAV.

39. The method according to claim 38, wherein the AAV virus capsid comprises a first member and a second cognate member of a specific binding pair covalently bound together.

40. The method according to claim 39, wherein the second cognate member is fused to a retargeting molecule.

41. The method according to claim 39, wherein the first member and second cognate member are a system selected from the group consisting of Spy Tag: Spy Catcher, Spy Tag002: SpyCatcher002, SpyTag003: SpyCatcher003, SnoopTag: SnoopCatcher, Isopeptag: Pilin-C, Isopeptag: Pilin-N, SnoopTagJr: SnoopCatcher, DogTag: DogCatcher, SdyTg: SdyCatcher, Jo: In, 3kptTag: 3kptCatcher, 40q1Taq/4oq1Catcher, NGTag/NGCatcher, Rumtrunk/Mooncake, Snoop ligase, GalacTag, Cpe, Ececo, and Corio.

42. The method according to claims 2-5, wherein the salt concentration during depth filtration is less than 100 mM.

43-47. (canceled)

48. A method of purifying full adeno-associated virus (AAV) capsids, wherein the method comprises the steps of
(a) subjecting a sample comprising AAV capsids to depth filtration to form a first pool;
(b) subjecting a first pool from step (a) to a single-pass tangential flow filtration to form a retentate comprising AAV capsids;
(c) subjecting the retentate to affinity capture to form a second pool comprising AAV capsids;
(d) subjecting the second pool to anion exchange chromatography in a buffer using a microstep or a linear gradient to form a third pool, wherein the third pool is enriched in the ratio of full capsids to capsids that are not full; and
(e) subjecting the third pool from step (d) to anion exchange chromatography in a buffer using a microstep or a linear gradient to form a fourth pool, wherein the fourth pool is further enriched in the ratio of full capsids to capsids that are not full.

49. The method according to claim 48, wherein the depth filtration does not require an endonuclease.

50. The method according to claim 48, wherein the single-pass tangential flow filtration uses a permeate pump.

51. The method according to claim 48, wherein the affinity capture further comprises non-AAV viral inactivation.

52-53. (canceled)
